(19) United States
(12) Patent Application Publication
(10) Pub. No.: US 2026/0242351 A1
(43) Pub. Date: Aug. 20, 2026

(54) LZK INHIBITORS

(71) Applicant: Uereka Biosciences, Inc., Lincolnshire, IL (US)

(72) Inventors: Erkan BALOGLU, Stoneham, MA (US); Soumya S. RAY, Quincy, MA (US)

(21) Appl. No.: 19/160,980

(22) PCT Filed: Feb. 28, 2024
(86) PCT No.: PCT/US2024/017745

§ 371 Date: Aug. 29, 2025

Related U.S. Application Data
(60) Provisional application No. 63/487,813, filed on Mar. 1, 2023.
(60) Provisional application No. 63/552,600, filed on Feb. 12, 2024.

(51) Int. Cl. C07D 401/12; A61K 31/497; A61K 31/5377; A61P 35/00; C07D 401/14; C07D 487/08

(52) U.S. Cl. CPC C07D 401/12; A61K 31/497; A61K 31/5377; A61P 35/00; C07D 401/14; C07D 487/08

(57) ABSTRACT

Described herein are LZK inhibitors and methods of utilizing these compounds in the treatment of cancer. Also described herein are pharmaceutical compositions containing such compounds.

### CROSS REFERENCE

This application claims benefit of U.S. Provisional Application No. 63/487,813, filed on Mar. 1, 2023, and U.S. Provisional Application No. 63/552,600, filed on Feb. 12, 2024, each of which is herein incorporated by reference in its entirety.

### BACKGROUND

Squamous cell carcinomas are known to develop in a broad range of anatomically distinct tumors including the head and neck, and esophagus. Collectively squamous cell tumors account for over two million incident cases per annum worldwide, with high morbidity and mortality rates due to a lack of precision therapeutic treatment options.

Currently, the standard of care for most squamous cell cancer patients, including head and neck and esophageal patients, remains limited. This is primarily due to a lack of actionable driver mutations that can be directly targeted with precision therapeutics. In addition to radiation and surgical interventions, the most common treatments include systemic therapies such as chemotherapeutics and/or checkpoint inhibitors (Johnson, D E, et al 2021, Nat Rev Dis Primers; 6(1): 92). The lack of efficacious and targeted treatment options for these patients has led researchers to seek to identify novel tumor drivers within incident patients which can be directly targeted with new therapeutic approaches.

### SUMMARY OF THE INVENTION

In one aspect, described herein is a compound of Formula (I), or a pharmaceutically acceptable salt or solvate thereof:
wherein:

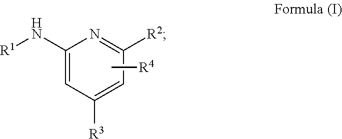

R¹ is a 6-membered heteroaryl ring optionally substituted with one, two, or three R⁵;
R² is —N(R¹⁰)(R¹¹), C₂₋₉heterocycloalkyl, —CH₂—C₂₋₉heterocycloalkyl, C₃₋₆cycloalkyl, or —CH₂—C₃₋₆cycloalkyl, wherein C₂₋₉heterocycloalkyl, —CH₂—C₂₋₉heterocycloalkyl, C₃₋₆cycloalkyl, and —CH₂—C₃₋₆cycloalkyl are optionally substituted with one, two, or three R⁶;
R³ is C₃₋₆cycloalkyl, C₂₋₉heterocycloalkyl, C₆₋₁₀aryl, or C₁₋₉heteroaryl, wherein each C₃₋₆cycloalkyl, C₂₋₉heterocycloalkyl, C₆₋₁₀aryl, and C₁₋₉heteroaryl are optionally substituted with one, two, or three R⁷;
R⁴ is selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀ aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R⁵ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰; each R⁶ is independently selected from halogen, oxo, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R⁷ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In another aspect, described herein is a compound, or a pharmaceutically acceptable salt or solvate thereof, having the structure of Formula (Ia):

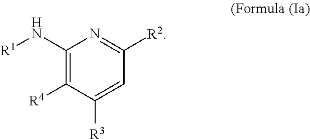

In another aspect, described herein is a compound, or a pharmaceutically acceptable salt or solvate thereof, having the structure of Formula (Ib).

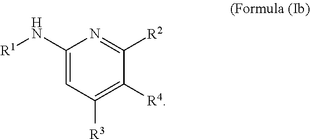

In some embodiments, is a compound of Formula (I), (Ia), or (Ib), or a pharmaceutically acceptable salt or solvate thereof, wherein R² is C₂₋₉heterocycloalkyl optionally substituted with one, two, or three R⁶.

In some embodiments, is a compound of Formula (I), (Ia), or (Ib), or a pharmaceutically acceptable salt or solvate thereof, wherein R² is selected from 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl, wherein 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl are optionally substituted with one, two, or three R⁶.

In some embodiments, is a compound of Formula (I), (Ia), or (Ib), or a pharmaceutically acceptable salt or solvate thereof, wherein each R⁶ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆cycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, is a compound of Formula (I), (Ia), or (Ib), or a pharmaceutically acceptable salt or solvate thereof, wherein each R⁶ is independently selected from halogen and C₁₋₆haloalkyl.

In some embodiments, is a compound of Formula (I), (Ia), or (Ib), or a pharmaceutically acceptable salt or solvate thereof, wherein R² is selected from

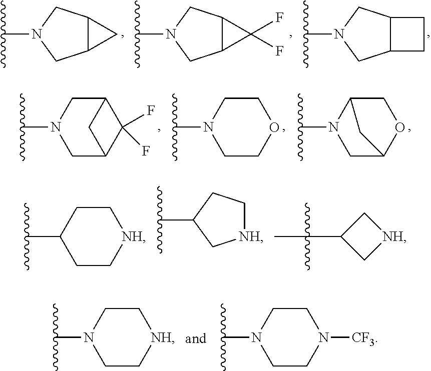

In some embodiments, is a compound of Formula (I), (Ia), or (Ib), or a pharmaceutically acceptable salt or solvate thereof, wherein R² is selected from

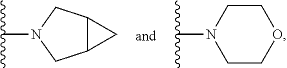 [SMILES: *N1CC2CC2C1 ; *N1CCOCC1]

In some embodiments, is a compound of Formula (I), (Ia), or (Ib), or a pharmaceutically acceptable salt or solvate thereof, wherein R³ is C₂₋₉heterocycloalkyl optionally substituted with one, two, or three R⁷.

In some embodiments, is a compound of Formula (I), (Ia), or (Ib), or a pharmaceutically acceptable salt or solvate thereof, wherein R³ is selected from 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl, wherein 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl are optionally substituted with one, two, or three R⁷.

In some embodiments, is a compound of Formula (I), (Ia), or (Ib), or a pharmaceutically acceptable salt or solvate thereof, wherein each R⁷ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆cycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, is a compound of Formula (I), (Ia), or (Ib), or a pharmaceutically acceptable salt or solvate thereof, wherein each R⁷ is independently selected from halogen, unsubstituted C₁₋₆alkyl, and unsubstituted —CH₂—C₃₋₆cycloalkyl.

In some embodiments, is a compound of Formula (I), (Ia), or (Ib), or a pharmaceutically acceptable salt or solvate thereof, wherein R³ is selected from

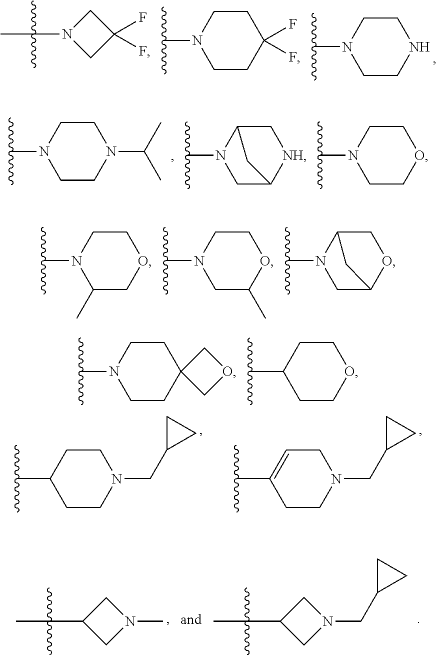

In some embodiments, is a compound of Formula (I), (Ia), or (Ib), or a pharmaceutically acceptable salt or solvate thereof, wherein R³ is selected from R³ is selected from

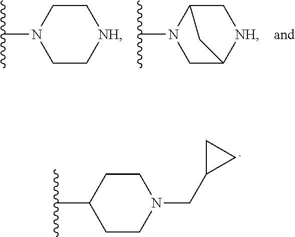

In some embodiments, is a compound of Formula (I), (Ia), or (Ib), or a pharmaceutically acceptable salt or solvate thereof, wherein R⁴ is selected from halogen, —CN, C₁₋₆alkyl, and C₁₋₆haloalkyl, wherein C₁₋₆alkyl and C₁₋₆haloalkyl are optionally substituted with one, two, or three groups selected from halogen, C₁₋₆alkyl, C₁₋₆haloalkyl, —OR¹⁰, and —N(R¹⁰)(R¹¹).

In some embodiments, is a compound of Formula (I), (Ia), or (Ib), or a pharmaceutically acceptable salt or solvate thereof, wherein R⁴ is halogen.

In some embodiments, is a compound of Formula (I), (Ia), or (Ib), or a pharmaceutically acceptable salt or solvate thereof, wherein R⁴ is —CN.

In some embodiments, is a compound of Formula (I), (Ia), or (Ib), or a pharmaceutically acceptable salt or solvate thereof, wherein R¹ is pyrazinyl or pyridinyl, wherein the pyrazinyl or pyridinyl are optionally substituted with one, two, or three R⁵.

In some embodiments, is a compound of Formula (I), (Ia), or (Ib), or a pharmaceutically acceptable salt or solvate thereof, wherein R¹ is pyrazinyl or pyridinyl, wherein the pyrazinyl are pyridinyl are optionally substituted with one R⁵.

In some embodiments, is a compound of Formula (I), (Ia), or (Ib), or a pharmaceutically acceptable salt or solvate thereof, wherein each R⁵ is independently selected from halogen, C₁₋₆alkyl, C₁₋₆haloalkyl, C₃₋₆cycloalkyl, and C₂₋₉heterocycloalkyl, wherein C₁₋₆alkyl, C₁₋₆haloalkyl, C₃₋₆cycloalkyl, and C₂₋₉heterocycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, is a compound of Formula (I), (Ia), or (Ib), or a pharmaceutically acceptable salt or solvate thereof, wherein R¹ is pyrazinyl or pyridinyl, wherein the pyrazinyl or pyridinyl are substituted with one R⁵ and R⁵ is cyclopropyl.

In some embodiments, is a compound of Formula (I), (Ia), or (Ib), or a pharmaceutically acceptable salt or solvate thereof, wherein R¹ is

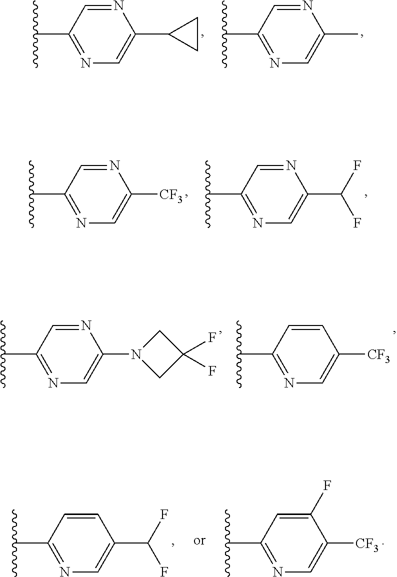

In some embodiments, is a compound of Formula (I), (Ia), or (Ib), or a pharmaceutically acceptable salt or solvate thereof, wherein R¹ is

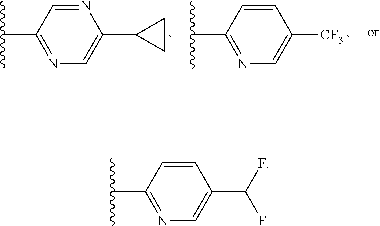

In another aspect, described herein is a pharmaceutical composition comprising a compound of Formula (I), (Ia), or (Ib), or a pharmaceutically acceptable salt or solvate thereof, and at least one pharmaceutically acceptable excipient.

In another aspect, described herein is a method of treating cancer in a mammal, comprising administering to the mammal a compound of Formula (I), (Ia), or (Ib), or a pharmaceutically acceptable salt or solvate thereof. In some embodiments, described herein is a method of treating cancer in a mammal, comprising administering to the mammal a compound of Formula (I), (Ia), or (Ib), or a pharmaceutically acceptable salt or solvate thereof, wherein the cancer is a cancer with LZK alterations. In some embodiments, described herein is a method of treating cancer in a mammal, comprising administering to the mammal a compound of Formula (I), (Ia), or (Ib), or a pharmaceutically acceptable salt or solvate thereof, wherein the cancer is selected from head and neck squamous cell carcinoma and esophageal squamous cell carcinoma.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

One common genetic alteration that is frequently observed in squamous cell tumors is that of gene copy number alterations. The most common conserved progenitor of gene copy number alterations is the distal amplification of chromosome 3 (3q26-3q29) (Derman B A, et al 2015, Transl. Lung Cancer Res; 4, 524-532) (Marur S, et all 2008, Mayo Clinic Proceedings; Vol 83, 489-501) (Abnet C C, et al 2018, Gastroenterology; 154, 360-373). This chromosome region contains many genes, including MAP3K13 which codes for the serine/threonine kinase Leucine Zipper Kinase (LZK). Notably, this genetic alteration is observed in greater than 20% of head and neck squamous cell patients, and greater than 20% of esophageal squamous cell patients (TCGA).

Emerging evidence has implicated MAP3K13 amplification, and the resulting overexpression of LZK, as a critical element for the survival of head and neck squamous cell tumor cell lines. Both genetic knockdown at the RNA level, and catalytic inhibition of the LZK protein result in anti-proliferative effects and tumor cell killing (Edwards Z C, et all 2017, Cancer Res; September 15; 77(18):4961-4972). The development of small molecule inhibitors targeting LZK activity represents a promising new avenue for a precision therapeutic treatment for patient harboring this mutation.

### Compounds

The compounds described herein, including pharmaceutically acceptable salts and solvates thereof, are leucine zipper-bearing kinase (LZK) inhibitors.

In one aspect, the present disclosure provides a compound of Formula (I), or a pharmaceutically acceptable salt or solvate thereof;

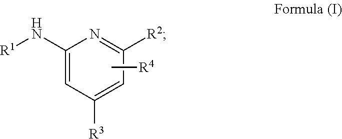

wherein;
R¹ is a 6-membered heteroaryl ring optionally substituted with one, two, or three R⁵;
R² is —N(R¹⁰)(R¹¹), C₂₋₉heterocycloalkyl, —CH₂—C₂₋₉heterocycloalkyl, C₃₋₆cycloalkyl, or —CH₂—C₃₋₆cycloalkyl, wherein C₂₋₉heterocycloalkyl, —CH₂—C₂₋₉heterocycloalkyl, C₃₋₆cycloalkyl, and —CH₂—C₃₋₆cycloalkyl are optionally substituted with one, two, or three R⁶;
R³ is C₃₋₆cycloalkyl, C₂₋₉heterocycloalkyl, C₆₋₁₀aryl, and C₁₋₉heteroaryl, wherein each C₃₋₆cycloalkyl, C₂₋₉heterocycloalkyl, C₆₋₁₀aryl, and C₁₋₉heteroaryl are optionally substituted with one, two, or three R⁷;
R⁴ is selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀ aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R⁵ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R⁶ is independently selected from halogen, oxo, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R⁷ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂- 6alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In some embodiments is a compound of Formula (I), or a pharmaceutically acceptable salt or solvate thereof, having the structure of Formula (Ia):

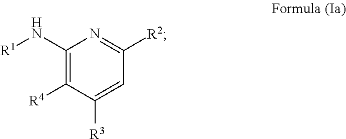

wherein R¹, R², R³, and R⁴ are each defined as in Formula (I).

In some embodiments is a compound of Formula (I), or a pharmaceutically acceptable salt or solvate thereof, having the structure of Formula (Ib):

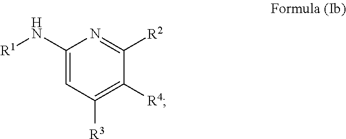

wherein R¹, R², R³, and R⁴ are each defined as in Formula (I).

In some embodiments, for a compound of Formula (I), (Ia), or (Ib), or a pharmaceutically acceptable salt or solvate thereof, R¹ is a 6-membered heteroaryl ring optionally substituted with one, two, or three R⁵.

In some embodiments, for a compound of Formula (I), (Ia), or (Ib), or a pharmaceutically acceptable salt or solvate thereof, R¹ is a 6-membered heteroaryl ring optionally substituted with one, two, or three R⁵; and
each R⁵ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀ aryl, and C₁₋₉heteroaryl.

In some embodiments, for a compound of Formula (I), (Ia), or (Ib), or a pharmaceutically acceptable salt or solvate thereof, R¹ is a 6-membered heteroaryl ring optionally substituted with one, two, or three R⁵; and
each R⁵ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —C(O)R¹³, —OC(O)R¹³, —C(O)N(R¹⁰)(R¹¹), —N(R¹²)C(O)R¹³, —CH₂C(O)N(R¹⁰)(R¹¹), —CH₂N(R¹²)C(O)R¹³, wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, hydroxy, C₁₋₆alkyl, C₁₋₆haloalkyl, and C₁₋₆alkoxy;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, and C₁₋₆alkoxy.

In some embodiments, for a compound of Formula (I), (Ia), or (Ib), or a pharmaceutically acceptable salt or solvate thereof, R¹ is a 6-membered heteroaryl ring optionally substituted with one, two, or three R⁵; and
each R⁵ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —C(O)OR¹⁰, —C(O)R¹³, —OC(O)R¹³, wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₃₋₆cycloalkyl, C₂₋₉heterocycloalkyl, C₆₋₁₀aryl, and C₁₋₉heteroaryl, wherein C₁₋₆alkyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, and C₁₋₆alkoxy;
each R¹¹ is independently selected from hydrogen, C₁₋₆alkyl, and C₁₋₆haloalkyl; and
each R¹³ is independently selected from C₁₋₆alkyl, C₃₋₆cycloalkyl, C₂₋₉heterocycloalkyl, C₆₋₁₀ aryl, and C₁₋₉heteroaryl, wherein C₁₋₆alkyl, C₃₋₆cycloalkyl, C₂₋₉heterocycloalkyl, C₆₋₁₀ aryl, and C₁₋₉heteroaryl are optionally substituted with one, two, or three groups selected from halogen, —CN, hydroxy, C₁₋₆alkyl, C₁₋₆haloalkyl, and C₁₋₆alkoxy.

In some embodiments, for a compound of Formula (I), (Ia), or (Ib), or a pharmaceutically acceptable salt or solvate thereof, R¹ is a 6-membered heteroaryl ring optionally substituted with one, two, or three R⁵; and
each R⁵ is independently selected from halogen, C₁₋₆alkyl, C₁₋₆haloalkyl, C₃₋₆cycloalkyl, —CH₂—C₃₋₆cycloalkyl, C₂₋₉heterocycloalkyl, —CH₂—C₂₋₉hetero cycloalkyl, C₆₋₁₀aryl, —CH₂—C₆₋₁₀aryl, C₁₋₉heteroaryl, —CH₂—C₁₋₉heteroaryl, wherein C₁₋₆ alkyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R¹ is a 6-membered heteroaryl ring optionally substituted with one, two, or three R⁵; and
each R⁵ is independently selected from halogen, C₁₋₆alkyl, C₁₋₆haloalkyl, C₃₋₆cycloalkyl, —CH₂—C₃₋₆cycloalkyl, C₂₋₉heterocycloalkyl, and —CH₂—C₂₋₉hetero cycloalkyl, wherein C₁₋₆alkyl, C₁₋₆haloalkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, R¹ is a 6-membered heteroaryl ring optionally substituted with one, two, or three R⁵; and
each R⁵ is independently selected from halogen, C₁₋₆alkyl, C₁₋₆haloalkyl, C₃₋₆cycloalkyl, and C₂₋₉heterocycloalkyl, wherein C₁₋₆alkyl, C₁₋₆haloalkyl, C₃₋₆cycloalkyl, and C₂₋₉heterocycloalkyl are optionally substituted with one, two, or three groups selected from halogen, C₁₋₆alkyl, C₁₋₆haloalkyl, —OR¹⁰, —N(R¹⁰)(R¹), and —C(O)OR¹⁰.

In some embodiments, for a compound of Formula (I), (Ia), or (Ib), or a pharmaceutically acceptable salt or solvate thereof, R¹ is a 6-membered heteroaryl ring optionally substituted with one, two, or three R⁵; and
each R⁵ is independently selected from halogen, C₁₋₆alkyl, C₁₋₆haloalkyl, C₃₋₆cycloalkyl, and C₂₋₉heterocycloalkyl, wherein C₁₋₆alkyl, C₁₋₆haloalkyl, C₃₋₆cycloalkyl, and C₂₋₉heterocycloalkyl are optionally substituted with one, two, or three groups selected from halogen, C₁₋₆alkyl, C₁₋₆haloalkyl, —OR¹⁰, —N(R¹⁰)(R¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R¹ is a 6-membered heteroaryl ring optionally substituted with one or two R⁵; and each R⁵ is independently selected from halogen, C₁₋₆alkyl, C₁₋₆ haloalkyl, C₃₋₆cycloalkyl, and C₂₋₉heterocycloalkyl, wherein C₁₋₆alkyl, C₁₋₆haloalkyl, C₃₋₆ cycloalkyl, and C₂₋₉heterocycloalkyl are optionally substituted with halogen. In some embodiments, R¹ is a 6-membered heteroaryl ring optionally substituted with one or two R⁵; and each R⁵ is independently selected from fluoro, —CH₃, —CHF₂, —CF₃, cyclopropyl, and azetidinyl, optionally substituted with one or two fluoro.

In some embodiments, for a compound of Formula (I), (Ia), or (Ib), or a pharmaceutically acceptable salt or solvate thereof, R¹ is a 6-membered heteroaryl ring optionally substituted with one R⁵; and R⁵ is independently selected from C₁₋₆haloalkyl and C₃₋₆cycloalkyl. In some embodiments, R¹ is a 6-membered heteroaryl ring optionally substituted with R⁵; and
each R⁵ is independently selected from —CHF₂, —CF₃, and cyclopropyl. In some embodiments, R¹ is a 6-membered heteroaryl ring optionally substituted with one R⁵; and R⁵ is cyclopropyl.

In some embodiments, for a compound of Formula (I), (Ia), or (Ib), or a pharmaceutically acceptable salt or solvate thereof, R¹ is pyrazinyl or pyridinyl, wherein the pyrazinyl or pyridinyl are optionally substituted with one, two, or three R⁵.

In some embodiments, for a compound of Formula (I), (Ia), or (Ib), or a pharmaceutically acceptable salt or solvate thereof, R¹ is pyrazinyl or pyridinyl, wherein the pyrazinyl or pyridinyl are optionally substituted with one, two, or three R⁵; and
each R⁵ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀ aryl, and C₁₋₉heteroaryl.

In some embodiments, for a compound of Formula (I), (Ia), or (Ib), or a pharmaceutically acceptable salt or solvate thereof, R¹ is pyrazinyl or pyridinyl, wherein the pyrazinyl or pyridinyl are optionally substituted with one, two, or three R⁵; and
each R⁵ is independently selected from halogen, C₁₋₆alkyl, C₁₋₆haloalkyl, C₃₋₆cycloalkyl, —CH₂—C₃₋₆cycloalkyl, C₂₋₉heterocycloalkyl, —CH₂—C₂₋₉hetero cycloalkyl, C₆₋₁₀aryl, —CH₂—C₆₋₁₀aryl, C₁₋₉heteroaryl, —CH₂—C₁₋₉heteroaryl, wherein C₁₋₆alkyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R¹ is pyrazinyl or pyridinyl, wherein the pyrazinyl or pyridinyl are optionally substituted with one, two, or three R⁵; and
each R⁵ is independently selected from halogen, C₁₋₆alkyl, C₁₋₆haloalkyl, C₃₋₆cycloalkyl, —CH₂—C₃₋₆cycloalkyl, C₂₋₉heterocycloalkyl, and —CH₂—C₂₋₉hetero cycloalkyl, wherein C₁₋₆alkyl, C₁₋₆haloalkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, R¹ is pyrazinyl or pyridinyl, wherein the pyrazinyl or pyridinyl are optionally substituted with one, two, or three R⁵; and
each R⁵ is independently selected from halogen, C₁₋₆alkyl, C₁₋₆haloalkyl, C₃₋₆cycloalkyl, and C₂₋₉heterocycloalkyl, wherein C₁₋₆alkyl, C₁₋₆haloalkyl, C₃₋₆cycloalkyl, and C₂₋₉heterocycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, R¹ is pyrazinyl or pyridinyl, wherein the pyrazinyl or pyridinyl are optionally substituted with one, two, or three R⁵; and
each R⁵ is independently selected from halogen, C₁₋₆alkyl, C₁₋₆haloalkyl, C₃₋₆cycloalkyl, and C₂₋₉heterocycloalkyl, wherein C₁₋₆alkyl, C₁₋₆haloalkyl, C₃₋₆cycloalkyl, and C₂₋₉heterocycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R¹ is pyrazinyl or pyridinyl, wherein the pyrazinyl or pyridinyl are optionally substituted with one or two R⁵; and each R⁵ is independently selected from halogen, C₁₋₆alkyl, C₁₋₆haloalkyl, C₃₋₆cycloalkyl, and C₂₋₉heterocycloalkyl, wherein C₁₋₆alkyl, C₁₋₆haloalkyl, C₃₋₆cycloalkyl, and C₂₋₉heterocycloalkyl are optionally substituted with halogen. In some embodiments, R¹ is pyrazinyl or pyridinyl, wherein the pyrazinyl or pyridinyl are optionally substituted with one or two R⁵; and each R⁵ is independently selected from fluoro, —CH₃, —CHF₂, —CF₃, cyclopropyl, and azetidinyl, optionally substituted with one or two fluoro.

In some embodiments, for a compound of Formula (I), (Ia), or (Ib), or a pharmaceutically acceptable salt or solvate thereof, R¹ is pyrazinyl or pyridinyl, wherein the pyrazinyl or pyridinyl are optionally substituted with one R⁵; and R⁵ is independently selected from C₁₋₆haloalkyl and C₃₋₆ cycloalkyl. In some embodiments, R¹ is pyrazinyl or pyridinyl, wherein the pyrazinyl or pyridinyl are optionally substituted with one R⁵. In some embodiments, R¹ is pyrazinyl or pyridinyl, wherein the pyrazinyl or pyridinyl are optionally substituted with one R⁵; and R⁵ is selected from —CHF₂, —CF₃, and cyclopropyl. In some embodiments, R¹ is pyrazinyl or pyridinyl, wherein the pyrazinyl or pyridinyl are optionally substituted with one R⁵; and R⁵ is cyclopropyl. In some embodiments, R¹ is pyrazinyl optionally substituted with one R⁵, and R⁵ is cyclopropyl or —CF₃. In some embodiments, R¹ is pyridinyl, optionally substituted with one R⁵, and R⁵ is —CF₃ or —CHF₂.

In some embodiments, for a compound of Formula (I), (Ia), or (Ib), or a pharmaceutically acceptable salt or solvate thereof, R¹ is

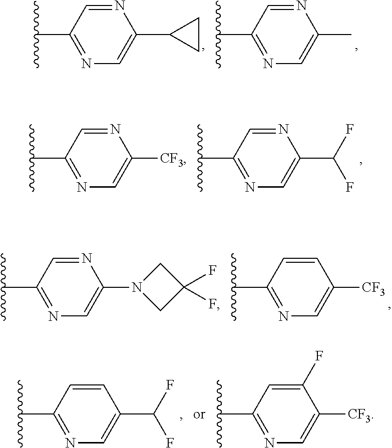

In some embodiments, for a compound of Formula (I), (Ia), or (Ib), or a pharmaceutically acceptable salt or solvate thereof, R¹ is

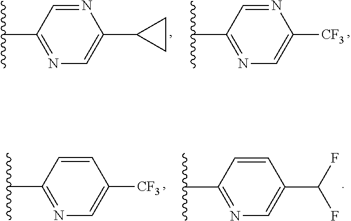

In some embodiments, for a compound of Formula (I), (Ia), or (Ib), or a pharmaceutically acceptable salt or solvate thereof, R¹ is

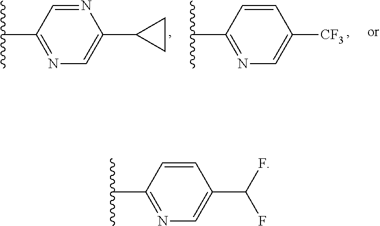

In some embodiments, for a compound of Formula (I), (Ia), or (Ib), or a pharmaceutically acceptable salt or solvate thereof, R¹ is

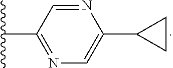 [SMILES: *c1cnc(C2CC2)cn1]

In some embodiments, for a compound of Formula (I), (Ia), or (Ib), or a pharmaceutically acceptable salt or solvate thereof, R¹ is

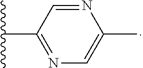 [SMILES: *c1cnc(C)cn1]

In some embodiments, for a compound of Formula (I), (Ia), or (Ib), or a pharmaceutically acceptable salt or solvate thereof, R¹ is

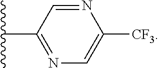 [SMILES: *c1cnc(C(F)(F)F)cn1]

In some embodiments, for a compound of Formula (I), (Ia), or (Ib), or a pharmaceutically acceptable salt or solvate thereof, R¹ is

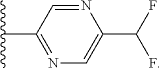 [SMILES: *c1cnc(C(F)F)cn1]

In some embodiments, for a compound of Formula (I), (Ia), or (Ib), or a pharmaceutically acceptable salt or solvate thereof, R¹ is

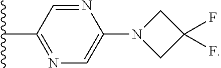 [SMILES: *c1cnc(N2CC(F)(F)C2)cn1]

In some embodiments, for a compound of Formula (I), (Ia), or (Ib), or a pharmaceutically acceptable salt or solvate thereof, R¹ is

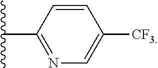 [SMILES: *c1ccc(C(F)(F)F)cn1]

In some embodiments, for a compound of Formula (I), (Ia), or (Ib), or a pharmaceutically acceptable salt or solvate thereof, R¹ is

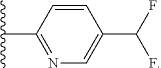 [SMILES: *c1ccc(C(F)F)cn1]

In some embodiments, for a compound of Formula (I), (Ia), or (Ib), or a pharmaceutically acceptable salt or solvate thereof, R¹ is

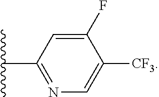 [SMILES: *c1cc(F)c(C(F)(F)F)cn1]

In some embodiments, for a compound of Formula (I), (Ia), or (Ib), or a pharmaceutically acceptable salt or solvate thereof, R² is —N(R¹⁰)(R¹¹), C₂₋₉heterocycloalkyl, —CH₂—C₂₋₉ heterocycloalkyl, C₃₋₆cycloalkyl, or —CH₂—C₃₋₆cycloalkyl, wherein C₂₋₉heterocycloalkyl, —CH₂—C₂₋₉heterocycloalkyl, C₃₋₆cycloalkyl, and —CH₂—C₃₋₆cycloalkyl are optionally substituted with one, two, or three R⁶.

In some embodiments, for a compound of Formula (I), (Ia), or (Ib), or a pharmaceutically acceptable salt or solvate thereof, R² is —N(R¹⁰)(R¹¹), C₂₋₉heterocycloalkyl, —CH₂—C₂₋₉heterocycloalkyl, C₃₋₆cycloalkyl, or —CH₂—C₃₋₆cycloalkyl, wherein C₂₋₉heterocycloalkyl, —CH₂—C₂₋₉heterocycloalkyl, C₃₋₆cycloalkyl, and —CH₂—C₃₋₆cycloalkyl are optionally substituted with one, two, or three R⁶; and
each R⁶ is independently selected from halogen, oxo, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀ aryl, and C₁₋₉heteroaryl.

In some embodiments, for a compound of Formula (I), (Ia), or (Ib), or a pharmaceutically acceptable salt or solvate thereof, R² is C₂₋₉heterocycloalkyl, —CH₂—C₂₋₉heterocycloalkyl, C₃₋₆ cycloalkyl, or —CH₂—C₃₋₆cycloalkyl, wherein C₂₋₉heterocycloalkyl, —CH₂—C₂₋₉heterocycloalkyl, C₃₋₆cycloalkyl, and —CH₂—C₃₋₆cycloalkyl are optionally substituted with one, two, or three R⁶; and
each R⁶ is independently selected from halogen, oxo, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —C(O)R¹³, —OC(O)R¹³, —C(O)N(R¹⁰)(R¹¹), —N(R¹²)C(O)R¹³, —CH₂C(O)N(R¹⁰)(R¹¹), —CH₂N(R¹²)C(O)R¹³, wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, hydroxy, C₁₋₆alkyl, C₁₋₆haloalkyl, and C₁₋₆alkoxy;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, and C₁₋₆alkoxy.

In some embodiments, for a compound of Formula (I), (Ia), or (Ib), or a pharmaceutically acceptable salt or solvate thereof, R² is C₂₋₉heterocycloalkyl, —CH₂—C₂₋₉heterocycloalkyl, C₃₋₆ cycloalkyl, or —CH₂—C₃₋₆cycloalkyl, wherein C₂₋₉heterocycloalkyl, —CH₂—C₂₋₉heterocycloalkyl, C₃₋₆cycloalkyl, and —CH₂—C₃₋₆cycloalkyl are optionally substituted with one, two, or three R⁶; and
each R⁶ is independently selected from halogen, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉ heterocycloalkyl, C₆₋₁₀aryl, —CH₂—C₆₋₁₀aryl, C₁₋₉heteroaryl, —CH₂—C₁₋₉heteroaryl, —OR¹⁰, —C(O)OR¹⁰, —C(O)R¹³, —OC(O)R¹³, wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₃₋₆cycloalkyl, C₂₋₉heterocycloalkyl, C₆₋₁₀aryl, and C₁₋₉heteroaryl, wherein C₁₋₆alkyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, and C₁₋₆alkoxy;
each R¹¹ is independently selected from hydrogen, C₁₋₆alkyl, and C₁₋₆haloalkyl; and
each R¹³ is independently selected from C₁₋₆alkyl, C₃₋₆cycloalkyl, C₂₋₉heterocycloalkyl, C₆₋₁₀ aryl, and C₁₋₉heteroaryl, wherein C₁₋₆alkyl, C₃₋₆cycloalkyl, C₂₋₉heterocycloalkyl, C₆₋₁₀ aryl, and C₁₋₉heteroaryl are optionally substituted with one, two, or three groups selected from halogen, —CN, hydroxy, C₁₋₆alkyl, C₁₋₆haloalkyl, and C₁₋₆alkoxy.

In some embodiments, for a compound of Formula (I), (Ia), or (Ib), or a pharmaceutically acceptable salt or solvate thereof, R² is C₂₋₉heterocycloalkyl, —CH₂—C₂₋₉heterocycloalkyl, C₃₋₆ cycloalkyl, or —CH₂—C₃₋₆cycloalkyl, wherein C₂₋₉heterocycloalkyl, —CH₂—C₂₋₉heterocycloalkyl, C₃₋₆cycloalkyl, and —CH₂—C₃₋₆cycloalkyl are optionally substituted with one, two, or three R⁶; and
each R⁶ is independently selected from halogen, C₁₋₆alkyl, C₁₋₆haloalkyl, C₃₋₆cycloalkyl, —CH₂—C₃₋₆cycloalkyl, C₂₋₉heterocycloalkyl-CH₂—C₂₋₉hetero cycloalkyl, C₆₋₁₀aryl, —CH₂—C₆₋₁₀aryl, C₁₋₉heteroaryl, —CH₂—C₁₋₉heteroaryl, wherein C₁₋₆alkyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R² is C₂₋₉heterocycloalkyl, —CH₂—C₂₋₉heterocycloalkyl, C₃₋₆ cycloalkyl, or —CH₂—C₃₋₆cycloalkyl, wherein C₂₋₉heterocycloalkyl, —CH₂—C₂₋₉ heterocycloalkyl, C₃₋₆cycloalkyl, and —CH₂—C₃₋₆cycloalkyl are optionally substituted with one, two, or three R⁶; and
each R⁶ is independently selected from halogen, C₁₋₆alkyl, C₁₋₆haloalkyl, C₃₋₆cycloalkyl, —CH₂—C₃₋₆cycloalkyl, C₂₋₉heterocycloalkyl, and —CH₂—C₂₋₉hetero cycloalkyl, wherein C₁₋₆alkyl, C₁₋₆haloalkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R² is C₂₋₉heterocycloalkyl, —CH₂—C₂₋₉heterocycloalkyl, C₃₋₆ cycloalkyl, or —CH₂—C₃₋₆cycloalkyl, wherein C₂₋₉heterocycloalkyl, —CH₂—C₂₋₉heterocycloalkyl, C₃₋₆cycloalkyl, and —CH₂—C₃₋₆cycloalkyl are optionally substituted with one, two, or three R⁶; and
each R⁶ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, R² is C₂₋₉heterocycloalkyl, —CH₂—C₂₋₉heterocycloalkyl, C₃₋₆ cycloalkyl, or —CH₂—C₃₋₆cycloalkyl, wherein C₂₋₉heterocycloalkyl, —CH₂—C₂₋₉heterocycloalkyl, C₃₋₆cycloalkyl, and —CH₂—C₃₋₆cycloalkyl are optionally substituted with one, two, or three R⁶; and
each R⁶ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R² is C₂₋₉heterocycloalkyl or C₃₋₆cycloalkyl, wherein C₂₋₉heterocycloalkyl and C₃₋₆cycloalkyl are optionally substituted with one, two, or three R⁶; and each R⁶ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, R² is C₂₋₉heterocycloalkyl or C₃₋₆cycloalkyl, wherein C₂₋₉heterocycloalkyl and C₃₋₆cycloalkyl are optionally substituted with one, two, or three R⁶; and each R⁶ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R² is C₂₋₉heterocycloalkyl optionally substituted with one, two, or three R⁶; and
each R⁶ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, R² is C₂₋₉heterocycloalkyl optionally substituted with one, two, or three R⁶; and
each R⁶ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R² is C₂₋₉heterocycloalkyl optionally substituted with one, two, or three R⁶; and each R⁶ is independently selected from halogen and C₁₋₆haloalkyl.

In some embodiments, for a compound of Formula (I), (Ia), or (Ib), or a pharmaceutically acceptable salt or solvate thereof, R² is selected from 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl, wherein 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl are optionally substituted with one, two, or three R⁶.

In some embodiments, for a compound of Formula (I), (Ia), or (Ib), or a pharmaceutically acceptable salt or solvate thereof, R² is selected from 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl, wherein 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl are optionally substituted with one, two, or three R⁶; and
each R⁶ is independently selected from halogen, oxo, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀ aryl, and C₁₋₉heteroaryl.

In some embodiments, for a compound of Formula (I), (Ia), or (Ib), or a pharmaceutically acceptable salt or solvate thereof, R² is selected from 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl, wherein 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl are optionally substituted with one, two, or three R⁶; and
each R⁶ is independently selected from halogen, C₁₋₆alkyl, C₁₋₆haloalkyl, C₃₋₆cycloalkyl, —CH₂—C₃₋₆cycloalkyl, C₂₋₉heterocycloalkyl-CH₂—C₂₋₉hetero cycloalkyl, C₆₋₁₀aryl, —CH₂—C₆₋₁₀aryl, C₁₋₉heteroaryl, —CH₂—C₁₋₉heteroaryl, wherein C₁₋₆alkyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R² is selected from 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl, wherein 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl are optionally substituted with one, two, or three R⁶; and
each R⁶ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, R² is selected from 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl, wherein 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl are optionally substituted with one, two, or three R⁶; and
each R⁶ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R² is selected from 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl, wherein 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl are optionally substituted with one, two, or three R⁶; and
each R⁶ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, R² is selected from 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl, wherein 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl are optionally substituted with one, two, or three R⁶; and
each R⁶ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R² is selected from 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl, wherein 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl are optionally substituted with one, two, or three R⁶; and each R⁶ is independently selected from halogen and C₁₋₆haloalkyl.

In some embodiments, for a compound of Formula (I), (Ia), or (Ib), or a pharmaceutically acceptable salt or solvate thereof, R² is selected from 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl. In some embodiments, R² is selected from 3-azabicyclo[3.1.0]hexanyl, 2-oxa-5-azabicyclo[2.2.1]heptanyl, morpholinyl, and piperidinyl.

In some embodiments, for a compound of Formula (I), (Ia), or (Ib), or a pharmaceutically acceptable salt or solvate thereof, R² is selected from

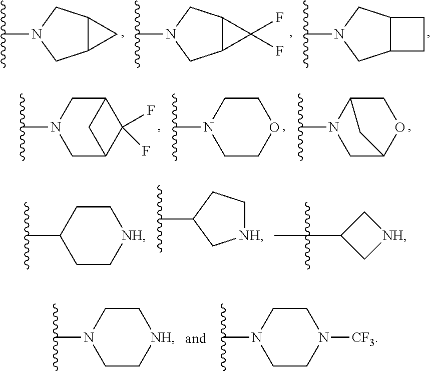

In some embodiments, for a compound of Formula (I), (Ia), or (Ib), or a pharmaceutically acceptable salt or solvate thereof, R² is selected from

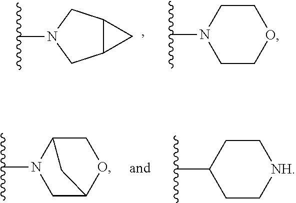

In some embodiments, for a compound of Formula (I), (Ia), or (Ib), or a pharmaceutically acceptable salt or solvate thereof, R² is

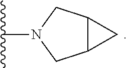 [SMILES: *N1CC2CC2C1]

In some embodiments, for a compound of Formula (I), (Ia), or (Ib), or a pharmaceutically acceptable salt or solvate thereof, R²

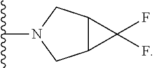 [SMILES: *N1CC2C(C1)C2(F)F]

In some embodiments, for a compound of Formula (I), (Ia), or (Ib), or a pharmaceutically acceptable salt or solvate thereof, R² is

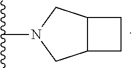 [SMILES: *N1CC2CCC2C1]

In some embodiments, for a compound of Formula (I), (Ia), or (Ib), or a pharmaceutically acceptable salt or solvate thereof, R² is

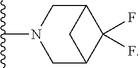 [SMILES: *N1CC2CC(C1)C2(F)F]

In some embodiments, for a compound of Formula (I), (Ia), or (Ib), or a pharmaceutically acceptable salt or solvate thereof, R² is

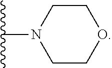 [SMILES: *N1CCOCC1]

In some embodiments, for a compound of Formula (I), (Ia), or (Ib), or a pharmaceutically acceptable salt or solvate thereof, R² is

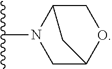 [SMILES: *N1CC2CC1CO2]

In some embodiments, for a compound of Formula (I), (Ia), or (Ib), or a pharmaceutically acceptable salt or solvate thereof, R² is

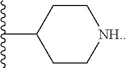 [SMILES: *C1CCNCC1]

In some embodiments, for a compound of Formula (I), (Ia), or (Ib), or a pharmaceutically acceptable salt or solvate thereof, R² is

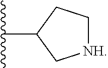 [SMILES: *C1CCNC1]

In some embodiments, for a compound of Formula (I), (Ia), or (Ib), or a pharmaceutically acceptable salt or solvate thereof, R² is

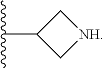 [SMILES: *C1CNC1]

In some embodiments, for a compound of Formula (I), (Ia), or (Ib), or a pharmaceutically acceptable salt or solvate thereof, R² is

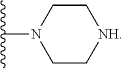 [SMILES: *N1CCNCC1]

In some embodiments, for a compound of Formula (I), (Ia), or (Ib), or a pharmaceutically acceptable salt or solvate thereof, R² is

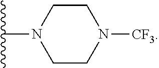 [SMILES: *N1CCN(C(F)(F)F)CC1]

In some embodiments, is a compound of Formula (I), (Ia), or (Ib), or a pharmaceutically acceptable salt or solvate thereof, wherein R² is selected from

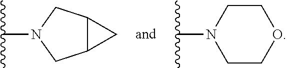 [SMILES: *N1CC2CC2C1 ; *N1CCOCC1]

In some embodiments, for a compound of Formula (I), (Ia), or (Ib), or a pharmaceutically acceptable salt or solvate thereof, R³ is C₃₋₆cycloalkyl, C₂₋₉heterocycloalkyl, C₆₋₁₀aryl, or C₁₋₉ heteroaryl, wherein each C₃₋₆cycloalkyl, C₂₋₉heterocycloalkyl, C₆₋₁₀aryl, and C₁₋₉heteroaryl are optionally substituted with one, two, or three R⁷.

In some embodiments, for a compound of Formula (I), (Ia), or (Ib), or a pharmaceutically acceptable salt or solvate thereof, R³ is C₃₋₆cycloalkyl, C₂₋₉heterocycloalkyl, C₆₋₁₀aryl, or C₁₋₉heteroaryl, wherein each C₃₋₆cycloalkyl, C₂₋₉heterocycloalkyl, C₆₋₁₀aryl, and C₁₋₉heteroaryl are optionally substituted with one, two, or three R⁷; and
each R⁷ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀ aryl, and C₁₋₉heteroaryl.

In some embodiments, for a compound of Formula (I), (Ia), or (Ib), or a pharmaceutically acceptable salt or solvate thereof, R³ is C₃₋₆cycloalkyl, C₂₋₉heterocycloalkyl, C₆₋₁₀aryl, or C₁₋₉ heteroaryl, wherein each C₃₋₆cycloalkyl, C₂₋₉heterocycloalkyl, C₆₋₁₀aryl, and C₁₋₉heteroaryl are optionally substituted with one, two, or three R⁷; and
each R⁷ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —C(O)R¹³, —OC(O)R¹³, —C(O)N(R¹⁰)(R¹¹), —N(R¹²)C(O)R¹³, —CH₂C(O)N(R¹⁰)(R¹¹), —CH₂N(R¹²)C(O)R¹³, wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, hydroxy, C₁₋₆alkyl, C₁₋₆haloalkyl, and C₁₋₆alkoxy;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, and C₁₋₆alkoxy.

In some embodiments, for the compound or salt of Formula (I), (Ia), or (Ib R³ is C₃₋₆cycloalkyl, C₂₋₉heterocycloalkyl, C₆₋₁₀aryl, or C₁₋₉heteroaryl, wherein each C₃₋₆cycloalkyl, C₂₋₉heterocycloalkyl, C₆₋₁₀aryl, and C₁₋₉heteroaryl are optionally substituted with one, two, or three R⁷; and
each R⁷ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —C(O)OR¹⁰, —C(O)R¹³, —OC(O)R¹³, wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₃₋₆cycloalkyl, C₂₋₉heterocycloalkyl, C₆₋₁₀aryl, and C₁₋₉heteroaryl, wherein C₁₋₆alkyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, and C₁₋₆alkoxy;
each R¹¹ is independently selected from hydrogen, C₁₋₆alkyl, and C₁₋₆haloalkyl; and
each R¹³ is independently selected from C₁₋₆alkyl, C₃₋₆cycloalkyl, C₂₋₉heterocycloalkyl, C₆₋₁₀ aryl, and C₁₋₉heteroaryl, wherein C₁₋₆alkyl, C₃₋₆cycloalkyl, C₂₋₉heterocycloalkyl, C₆₋₁₀ aryl, and C₁₋₉heteroaryl are optionally substituted with one, two, or three groups selected from halogen, —CN, hydroxy, C₁₋₆alkyl, C₁₋₆haloalkyl, and C₁₋₆alkoxy.

In some embodiments, for a compound of Formula (I), (Ia), or (Ib), or a pharmaceutically acceptable salt or solvate thereof, R³ is C₃₋₆cycloalkyl, C₂₋₉heterocycloalkyl, C₆₋₁₀aryl, or C₁₋₉ heteroaryl, wherein each C₃₋₆cycloalkyl, C₂₋₉heterocycloalkyl, C₆₋₁₀aryl, and C₁₋₉heteroaryl are optionally substituted with one, two, or three R⁷; and
each R⁷ is independently selected from halogen, —C₁₋₆alkyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl, wherein C₁₋₆alkyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀ aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R³ is C₃₋₆cycloalkyl or C₂₋₉heterocycloalkyl, each optionally substituted with one, two, or three R⁷; and
each R⁷ is independently selected from halogen, C₁₋₆alkyl, C₁₋₆haloalkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₁₋₆haloalkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, R³ is C₃₋₆cycloalkyl or C₂₋₉heterocycloalkyl, each optionally substituted with one, two, or three R⁷; and
each R⁷ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl, are optionally substituted with one, two, or three groups selected from halogen, C₁₋₆alkyl, C₁₋₆haloalkyl, —OR¹⁰, —N(R¹⁰)(R¹), and —C(O)OR¹⁰.

In some embodiments, for a compound of Formula (I), (Ia), or (Ib), or a pharmaceutically acceptable salt or solvate thereof, R³ is C₃₋₆cycloalkyl or C₂₋₉heterocycloalkyl, each optionally substituted with one, two, or three R⁷; and
each R⁷ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R³ is C₂₋₉heterocycloalkyl optionally substituted with one, two, or three R⁷.

In some embodiments, for a compound of Formula (I), (Ia), or (Ib), or a pharmaceutically acceptable salt or solvate thereof, R³ is C₂₋₉heterocycloalkyl optionally substituted with one, two, or three R⁷; and
each R⁷ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, R³ is C₂₋₉heterocycloalkyl optionally substituted with one, two, or three R⁷; and
each R⁷ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R³ is C₂₋₉heterocycloalkyl optionally substituted with one or two R⁷; and each R⁷ is independently selected from halogen, C₁₋₆alkyl, and —CH₂—C₃₋₆cycloalkyl. In some embodiments, R³ is C₂₋₉heterocycloalkyl optionally substituted with one, two, or three R⁷; and each R⁷ is independently selected from halogen, unsubstituted C₁₋₆alkyl, and unsubstituted —CH₂—C₃₋₆cycloalkyl. In some embodiments, R³ is C₂₋₉heterocycloalkyl optionally substituted with one or two R⁷; and each R⁷ is independently selected from halogen, unsubstituted C₁₋₆alkyl, and unsubstituted —CH₂—C₃₋₆cycloalkyl. In some embodiments, R³ is C₂₋₉heterocycloalkyl optionally substituted with one or two R⁷; and each R⁷ is independently selected from fluoro, —CH₃, and —CH₂— cyclopropyl. In some embodiments, R³ is C₂₋₉heterocycloalkyl optionally substituted with one or two R⁷; and each R⁷ is independently selected from fluoro and —CH₂-cyclopropyl.

In some embodiments, for a compound of Formula (I), (Ia), or (Ib), or a pharmaceutically acceptable salt or solvate thereof, R³ is selected from 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl, wherein 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl are optionally substituted with one, two, or three R⁷.

In some embodiments, for a compound of Formula (I), (Ia), or (Ib), or a pharmaceutically acceptable salt or solvate thereof, R³ is selected from 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl, wherein 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl are optionally substituted with one, two, or three R⁷; and
each R⁷ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀ aryl, and C₁₋₉heteroaryl.

In some embodiments, for a compound of Formula (I), (Ia), or (Ib), or a pharmaceutically acceptable salt or solvate thereof, R³ is selected from 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl, wherein 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl are optionally substituted with one, two, or three R⁷; and
each R⁷ is independently selected from halogen, —C₁₋₆alkyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl, wherein C₁₋₆alkyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀ aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R³ is selected from 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl, wherein 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl are optionally substituted with one, two, or three R⁷; and
each R⁷ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, R³ is selected from 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl, wherein 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl are optionally substituted with one, two, or three R⁷; and
each R⁷ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R³ is selected from 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl, wherein 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl are optionally substituted with one, two, or three R⁷.

In some embodiments, for a compound of Formula (I), (Ia), or (Ib), or a pharmaceutically acceptable salt or solvate thereof, R³ is selected from 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl, wherein 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl are optionally substituted with one, two, or three R⁷; and
each R⁷ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆ cycloalkyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R³ is selected from R³ is selected from 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl, wherein 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl are optionally substituted with one or two R⁷; and
each R⁷ is independently selected from halogen, C₁₋₆alkyl, and —CH₂—C₃₋₆cycloalkyl. In some embodiments, R³ is selected from 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl, wherein 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl are optionally substituted with one, two, or three R⁷; and each R⁷ is independently selected from halogen, unsubstituted C₁₋₆alkyl, and unsubstituted —CH₂—C₃₋₆ cycloalkyl. In some embodiments, R³ is selected from 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl, wherein 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl are optionally substituted with one or two R⁷; and each R⁷ is independently selected from halogen, unsubstituted C₁₋₆alkyl, and unsubstituted —CH₂—C₃₋₆cycloalkyl.

In some embodiments, for a compound of Formula (I), (Ia), or (Ib), or a pharmaceutically acceptable salt or solvate thereof, R³ is selected from 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl, wherein 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl are optionally substituted with one or two R⁷; and each R⁷ is independently selected from fluoro, —CH₃, and —CH₂-cyclopropyl. In some embodiments, R³ is selected 2,5 -diazabicyclo[2.2.1]heptane, 2-oxa-5-azabicyclo[2.2.1]heptane, morpholinyl, azetidinyl, and piperazinyl, wherein 2,5-diazabicyclo[2.2.1]heptane, 2-oxa-5-azabicyclo[2.2.1]heptane, morpholinyl, azetidinyl, and piperazinyl are optionally substituted with one or two R⁷; and each R⁷ is independently selected from fluoro and —CH₂-cyclopropyl.

In some embodiments, for a compound of Formula (I), (Ia), or (Ib), or a pharmaceutically acceptable salt or solvate thereof, R³ is selected from

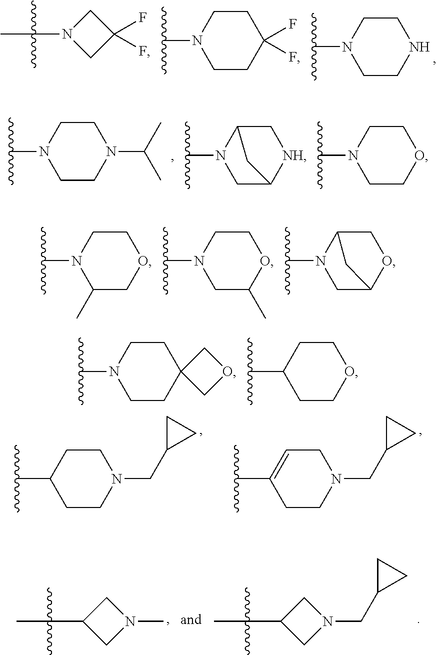

In some embodiments, for a compound of Formula (I), (Ia), or (Ib), or a pharmaceutically acceptable salt or solvate thereof, R³ is selected from

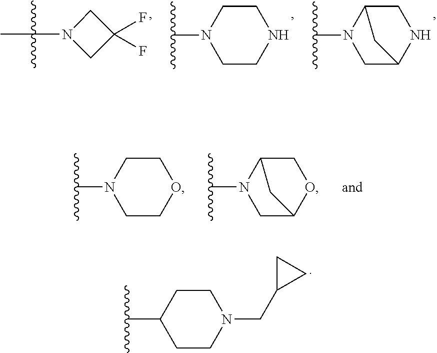

In some embodiments, is a compound of Formula (I), (Ia), or (Ib), or a pharmaceutically acceptable salt or solvate thereof, wherein R³ is selected from R³ is selected from

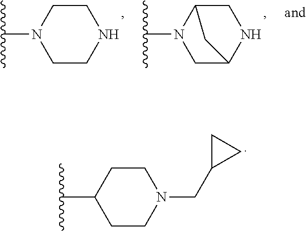

In some embodiments, for a compound of Formula (I), (Ia), or (Ib), or a pharmaceutically acceptable salt or solvate thereof, R³ is

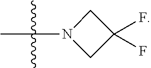 [SMILES: *N1CC(F)(F)C1]

In some embodiments, for a compound of Formula (I), (Ia), or (Ib), or a pharmaceutically acceptable salt or solvate thereof, R³ is

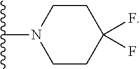 [SMILES: *N1CCC(F)(F)CC1]

In some embodiments, for a compound of Formula (I), (Ia), or (Ib), or a pharmaceutically acceptable salt or solvate thereof, R³ is

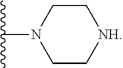 [SMILES: *N1CCNCC1]

In some embodiments, for a compound of Formula (I), (Ia), or (Ib), or a pharmaceutically acceptable salt or solvate thereof, R³ is

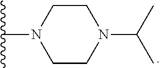 [SMILES: *N1CCN(C(C)C)CC1]

In some embodiments, for a compound of Formula (I), (Ia), or (Ib), or a pharmaceutically acceptable salt or solvate thereof, R³ is

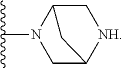 [SMILES: *N1CC2CC1CN2]

In some embodiments, for a compound of Formula (I), (Ia), or (Ib), or a pharmaceutically acceptable salt or solvate thereof, R³ is

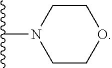 [SMILES: *N1CCOCC1]

In some embodiments, for a compound of Formula (I), (Ia), or (Ib), or a pharmaceutically acceptable salt or solvate thereof, R³ is

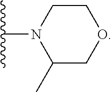 [SMILES: *N1CCOCC1C]

In some embodiments, for a compound of Formula (I), (Ia), or (Ib), or a pharmaceutically acceptable salt or solvate thereof, R³ is

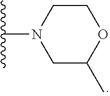 [SMILES: *N1CCOC(C)C1]

In some embodiments, for a compound of Formula (I), (Ia), or (Ib), or a pharmaceutically acceptable salt or solvate thereof, R³ is

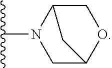 [SMILES: *N1CC2CC1CO2]

In some embodiments, for a compound of Formula (I), (Ia), or (Ib), or a pharmaceutically acceptable salt or solvate thereof, R³ is

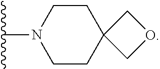 [SMILES: *N1CCC2(CC1)COC2]

In some embodiments, for a compound of Formula (I), (Ia), or (Ib), or a pharmaceutically acceptable salt or solvate thereof, R³ is

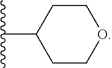 [SMILES: *C1CCOCC1]

In some embodiments, for a compound of Formula (I), (Ia), or (Ib), or a pharmaceutically acceptable salt or solvate thereof, R³ is

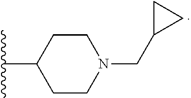 [SMILES: *C1CCN(CC2CC2)CC1]

In some embodiments, for a compound of Formula (I), (Ia), or (Ib), or a pharmaceutically acceptable salt or solvate thereof, R³ is

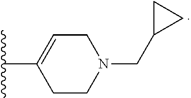 [SMILES: *C1=CCN(CC2CC2)CC1]

In some embodiments, for a compound of Formula (I), (Ia), or (Ib), or a pharmaceutically acceptable salt or solvate thereof, R³ is

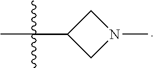 [SMILES: *C1CN(C)C1]

In some embodiments, for a compound of Formula (I), (Ia), or (Ib), or a pharmaceutically acceptable salt or solvate thereof, R³ is

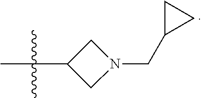 [SMILES: *C1CN(CC2CC2)C1]

In some embodiments, for a compound of Formula (I), (Ia), or (Ib), or a pharmaceutically acceptable salt or solvate thereof, R⁴ is selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, and —N(R¹⁰)(R¹¹); and
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀ aryl, and C₁₋₉heteroaryl.

In some embodiments, for a compound of Formula (I), (Ia), or (Ib), or a pharmaceutically acceptable salt or solvate thereof, R⁴ is selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —C(O)R¹³, —OC(O)R¹³, wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆ haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₃₋₆cycloalkyl, C₂₋₉heterocycloalkyl, C₆₋₁₀aryl, and C₁₋₉heteroaryl, wherein C₁₋₆alkyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, and C₁₋₆alkoxy;
each R¹¹ is independently selected from hydrogen, C₁₋₆alkyl, and C₁₋₆haloalkyl; and
each R¹³ is independently selected from C₁₋₆alkyl, C₃₋₆cycloalkyl, C₂₋₉heterocycloalkyl, C₆₋₁₀ aryl, and C₁₋₉heteroaryl, wherein C₁₋₆alkyl, C₃₋₆cycloalkyl, C₂₋₉heterocycloalkyl, C₆₋₁₀ aryl, and C₁₋₉heteroaryl are optionally substituted with one, two, or three groups selected from halogen, —CN, hydroxy, C₁₋₆alkyl, C₁₋₆haloalkyl, and C₁₋₆alkoxy.

In some embodiments, for a compound of Formula (I), (Ia), or (Ib), or a pharmaceutically acceptable salt or solvate thereof, R⁴ is selected from halogen, —CN, C₁₋₆alkyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, —OR¹⁰, and —N(R¹⁰)(R¹¹), wherein C₁₋₆alkyl, C₁₋₆haloalkyl, C₃₋₆cycloalkyl, —CH₂—C₃₋₆cycloalkyl, C₂₋₉heterocycloalkyl, and —CH₂—C₂₋₉heterocycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R⁴ is selected from halogen, —CN, C₁₋₆alkyl, C₁₋₆haloalkyl, —OR¹⁰, and —N(R¹⁰)(R¹¹), wherein C₁₋₆alkyl, and C₁₋₆haloalkyl are optionally substituted with one, two, or three groups selected from halogen, C₁₋₆alkyl, C₁₋₆haloalkyl, —OR¹⁰, —N(R¹⁰)(R¹), and —C(O)OR¹⁰.

In some embodiments, for a compound of Formula (I), (Ia), or (Ib), or a pharmaceutically acceptable salt or solvate thereof, R⁴ is selected from halogen, —CN, C₁₋₆alkyl, C₁₋₆haloalkyl, —OR¹⁰, and —N(R¹⁰)(R¹), wherein C₁₋₆alkyl, and C₁₋₆haloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, and C₁₋₆ haloalkyl; and
each R¹¹ is independently selected from hydrogen, C₁₋₆alkyl, and C₁₋₆haloalkyl.

In some embodiments, for a compound of Formula (I), (Ia), or (Ib), or a pharmaceutically acceptable salt or solvate thereof, R⁴ is selected from halogen, —CN, C₁₋₆alkyl, and C₁₋₆haloalkyl, wherein C₁₋₆alkyl, and C₁₋₆haloalkyl are optionally substituted with one, two, or three groups selected from halogen, C₁₋₆alkyl, C₁₋₆haloalkyl, —OR¹⁰, and —N(R¹⁰)(R¹).

In some embodiments, for a compound of Formula (I), (Ia), or (Ib), or a pharmaceutically acceptable salt or solvate thereof, R⁴ is selected from halogen, —CN, C₁₋₆alkyl, and C₁₋₆haloalkyl, wherein C₁₋₆alkyl, and C₁₋₆haloalkyl are optionally substituted with one, two, or three groups selected from halogen, C₁₋₆alkyl, C₁₋₆haloalkyl, —OR¹⁰, and —N(R¹⁰)(R¹);
each R¹⁰ is independently selected from hydrogen, and C₁₋₆alkyl; and
each R¹¹ is independently selected from hydrogen, and C₁₋₆alkyl.

In some embodiments, for a compound of Formula (I), (Ia), or (Ib), or a pharmaceutically acceptable salt or solvate thereof, R⁴ is selected from halogen, —CN, C₁₋₆alkyl, —OR¹⁰, and —N(R¹⁰)(R¹¹), wherein C₁₋₆alkyl is optionally substituted with one, two, or three groups selected from halogen and C₁₋₆alkyl. In some embodiments, R⁴ is selected from halogen, —CN, C₁₋₆alkyl, —OR¹⁰, and —N(R¹⁰)(R¹¹).

In some embodiments, for a compound of Formula (I), (Ia), or (Ib), or a pharmaceutically acceptable salt or solvate thereof, R⁴ is selected from halogen, —CN, C₁₋₆alkyl, —OR¹⁰, and —N(R¹⁰)(R¹¹), wherein C₁₋₆alkyl, and C₁₋₆haloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, and C₁₋₆alkyl; and
each R¹¹ is hydrogen.

In some embodiments, for a compound of Formula (I), (Ia), or (Ib), or a pharmaceutically acceptable salt or solvate thereof, R⁴ is selected from halogen, —CN, C₁₋₆alkyl, —OR¹⁰, and —N(R¹⁰)(R¹¹); and
each R¹⁰ is independently selected from hydrogen, and C₁₋₆alkyl; and
each R¹¹ is hydrogen.

In some embodiments, for a compound of Formula (I), (Ia), or (Ib), or a pharmaceutically acceptable salt or solvate thereof, R⁴ is selected from halogen, —CN, C₁₋₆alkyl, —OR¹⁰, and —N(R¹⁰)(R¹¹); and
each R¹⁰ is independently selected from hydrogen, and —CH₃; and
each R¹¹ is hydrogen.

In some embodiments, for a compound of Formula (I), (Ia), or (Ib), or a pharmaceutically acceptable salt or solvate thereof, R⁴ is selected from halogen, —CN, C₁₋₆alkyl, —OH, —OCH₃ and —NH₂. In some embodiments, R⁴ is selected from fluoro, chloro, —CN, —CH₃, isopropyl, —OH, —OCH₃ and —NH₂. In some embodiments, R⁴ is halogen. In some embodiments, R⁴ is selected from fluoro and chloro. In some embodiments, R⁴ is fluoro. In some embodiments, R⁴ is chloro. In some embodiments, for a compound of Formula (I), (Ia), or (Ib), or a pharmaceutically acceptable salt or solvate thereof, R⁴ is —CN. In some embodiments, for a compound of Formula (I), (Ia), or (Ib), or a pharmaceutically acceptable salt or solvate thereof, R⁴ is C₁₋₆alkyl. In some embodiments, for a compound of Formula (I), (Ia), or (Ib), or a pharmaceutically acceptable salt or solvate thereof, R⁴ is —CH₃. In some embodiments, for a compound of Formula (I), (Ia), or (Ib), or a pharmaceutically acceptable salt or solvate thereof, R⁴ is —CH₂CH₃. In some embodiments, for a compound of Formula (I), (Ia), or (Ib), or a pharmaceutically acceptable salt or solvate thereof, R⁴ is —CH(CH₃)₂. In some embodiments, for a compound of Formula (I), (Ia), or (Ib), or a pharmaceutically acceptable salt or solvate thereof, R⁴ is —OH. In some embodiments, for a compound of Formula (I), (Ia), or (Ib), or a pharmaceutically acceptable salt or solvate thereof, R⁴ is —OCH₃. In some embodiments, for a compound of Formula (I), (Ia), or (Ib), or a pharmaceutically acceptable salt or solvate thereof, R⁴ is —NH₂.

In some embodiments, for a compound of Formula (I), (Ia), or (Ib), or a pharmaceutically acceptable salt or solvate thereof, each R⁵ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, each R⁵ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —C(O)OR¹⁰, —C(O)R¹³, —OC(O)R¹³, wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, each R⁵ is independently selected from halogen, C₁₋₆alkyl, C₁₋₆haloalkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₁₋₆haloalkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, each R⁵ is independently selected from halogen, C₁₋₆alkyl, C₁₋₆ haloalkyl, C₃₋₆cycloalkyl, and C₂₋₉heterocycloalkyl, wherein C₁₋₆alkyl, C₁₋₆haloalkyl, C₃₋₆cycloalkyl, and C₂₋₉heterocycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰. In some embodiments, each R⁵ is independently selected from halogen, C₁₋₆alkyl, C₁₋₆haloalkyl, C₃₋₆ cycloalkyl, and C₂₋₉heterocycloalkyl, wherein C₁₋₆alkyl, C₁₋₆haloalkyl, C₃₋₆cycloalkyl, and C₂₋₉heterocycloalkyl are optionally substituted with halogen. In some embodiments, each R⁵ is independently selected from fluoro, —CH₃, —CHF₂, —CF₃, cyclopropyl, and azetidinyl, wherein cyclopropyl and azetidinyl are optionally substituted with one or two fluoro. In some embodiments, each R⁵ is independently selected from —CHF₂, —CF₃, and cyclopropyl. In some embodiments, R⁵ is cyclopropyl.

In some embodiments, for a compound of Formula (I), (Ia), or (Ib), or a pharmaceutically acceptable salt or solvate thereof, each R⁶ is independently selected from halogen, oxo, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀ aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, each R⁶ is independently selected from halogen, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —C(O)OR¹⁰, —C(O)R¹³, —OC(O)R¹³, wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, each R⁶ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆cycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰. In some embodiments, each R⁶ is independently selected from halogen and C₁₋₆haloalkyl. In some embodiments, each R⁶ is independently selected from halogen. In some embodiments, each R⁶ is independently selected from C₁₋₆haloalkyl.

In some embodiments, for a compound of Formula (I), (Ia), or (Ib), or a pharmaceutically acceptable salt or solvate thereof, each R⁷ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, each R⁷ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —C(O)OR¹⁰, —C(O)R¹³, —OC(O)R¹³, wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, each R⁷ is independently selected from halogen, C₁₋₆alkyl, C₁₋₆haloalkyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₁₋₆haloalkyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

In some embodiments, for a compound of Formula (I), (Ia), or (Ib), or a pharmaceutically acceptable salt or solvate thereof, each R⁷ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆cycloalkyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰. In some embodiments, each R⁷ is independently selected from halogen, C₁₋₆alkyl, and —CH₂—C₃₋₆cycloalkyl. In some embodiments, each R⁷ is independently selected from halogen, unsubstituted C₁₋₆alkyl, and unsubstituted —CH₂—C₃₋₆cycloalkyl. In some embodiments, each R⁷ is independently selected from fluoro, —CH₃, and —CH₂-cyclopropyl. In some embodiments, each R⁷ is independently selected from fluoro and —CH₂-cyclopropyl. In some embodiments, each R⁷ is fluoro. In some embodiments, each R⁷ is —CH₂-cyclopropyl.

In some embodiments, provided herein is a compound of Formula (I), (Ia), or (Ib), or a pharmaceutically acceptable salt thereof, having the structure:

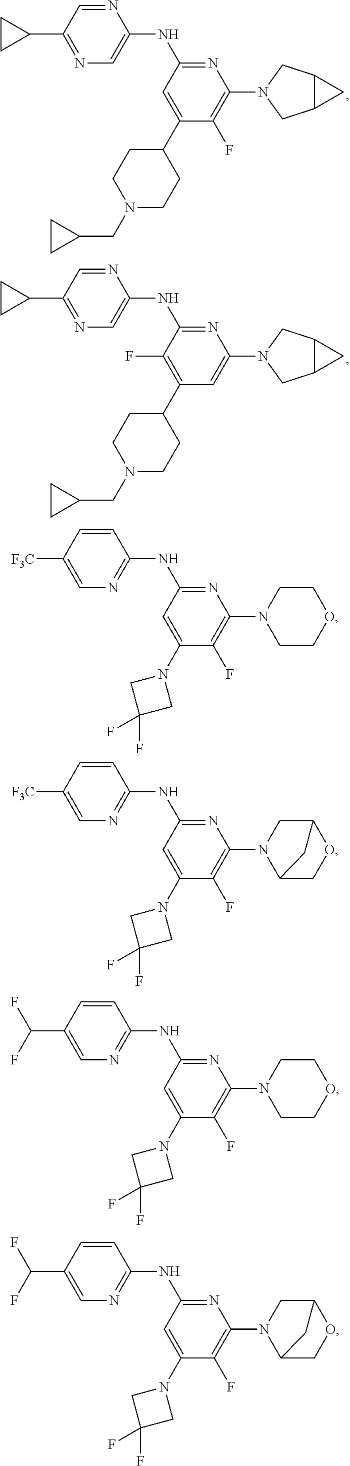

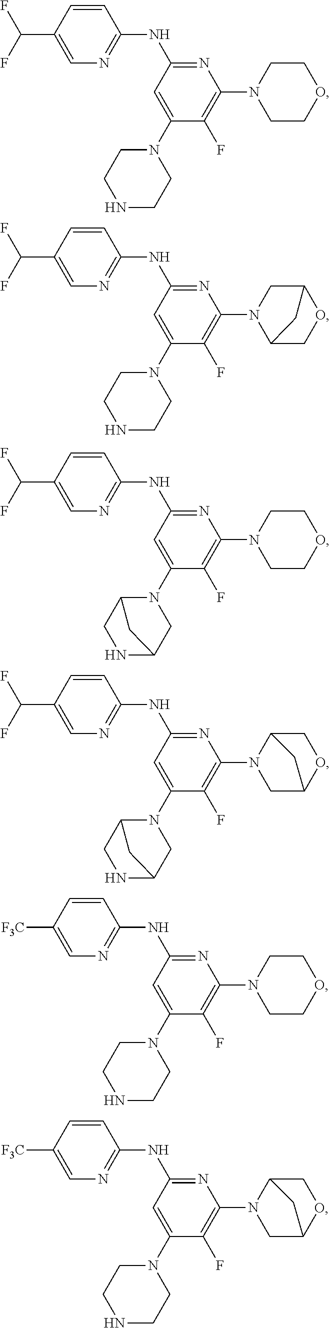

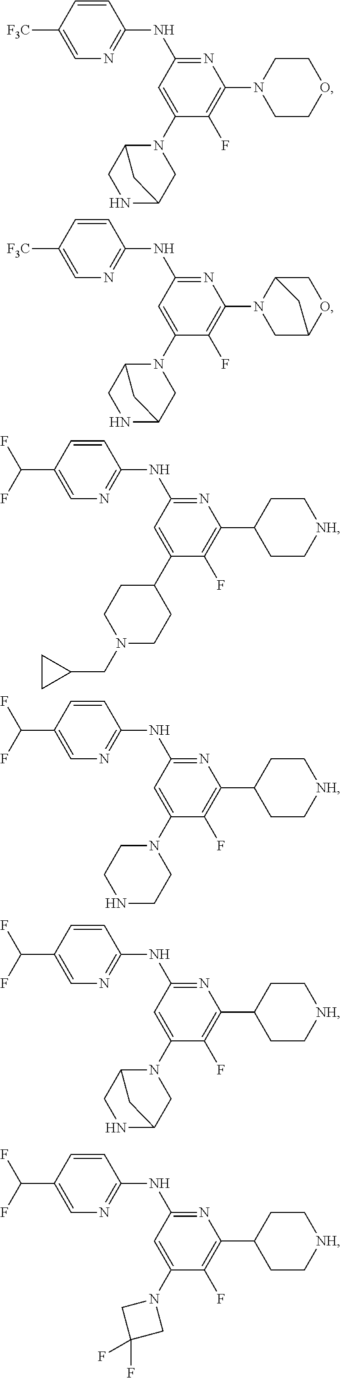

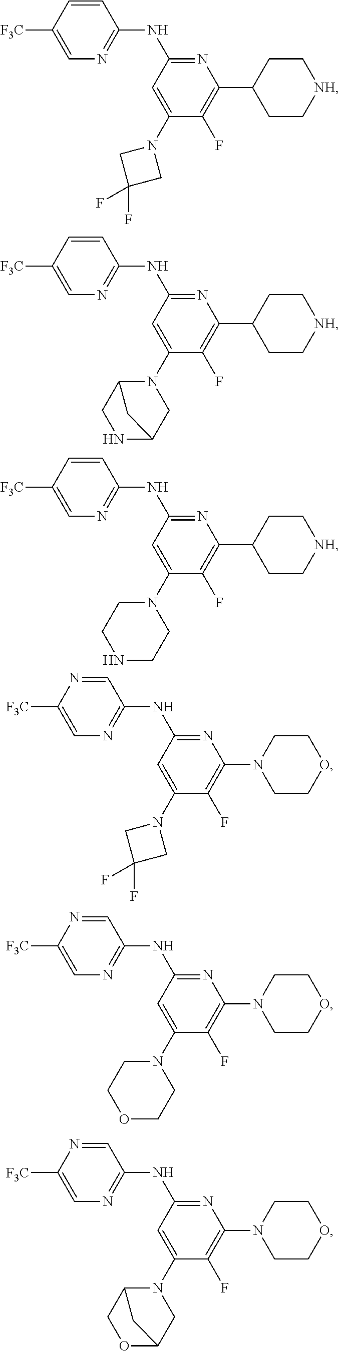

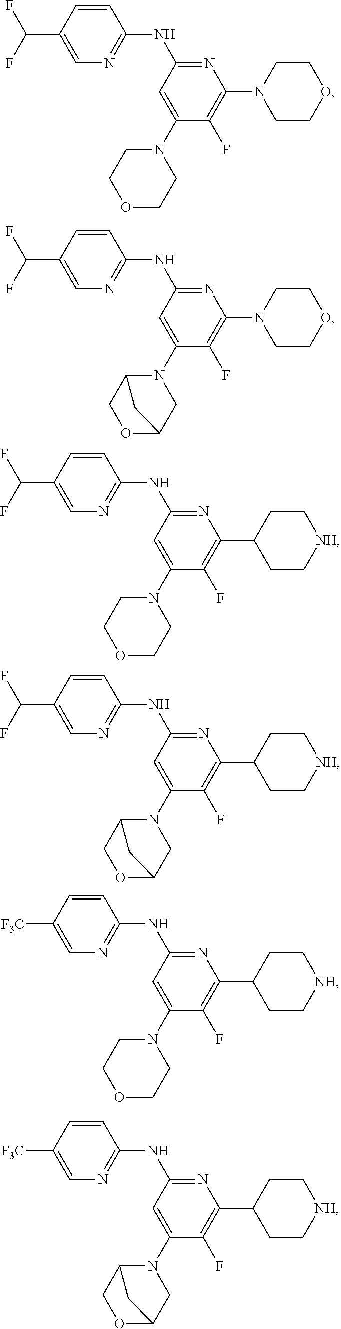

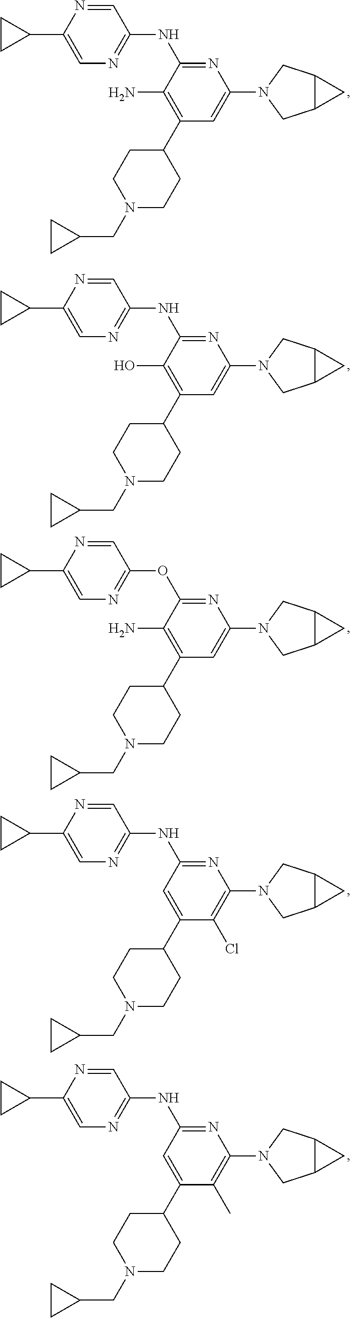

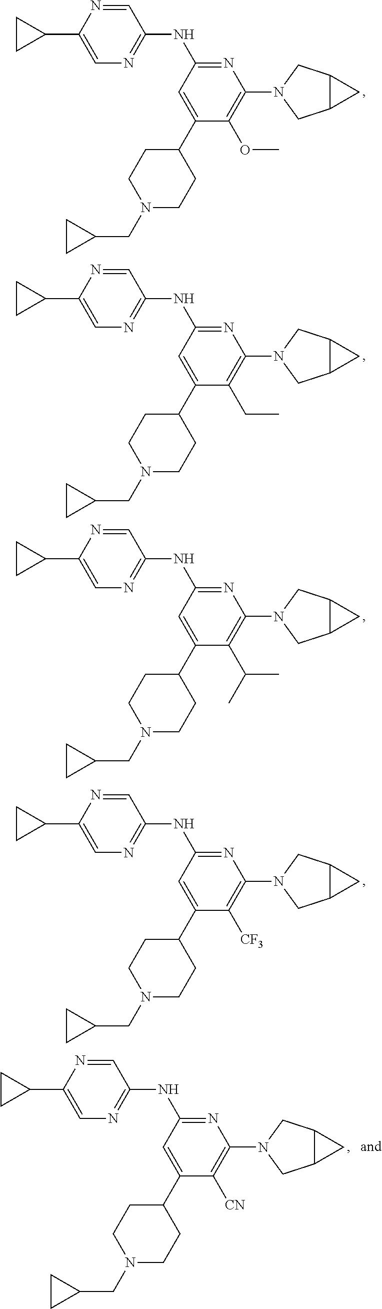

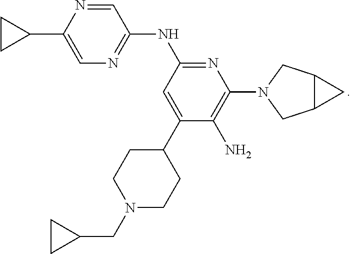 [SMILES: Nc1c(C2CCN(CC3CC3)CC2)cc(Nc2cnc(C3CC3)cn2)nc1N1CC2CC2C1]

In some embodiments, provided herein is a compound of Formula (Ia), or (Ib), or a pharmaceutically acceptable salt or solvate thereof, having the structure:

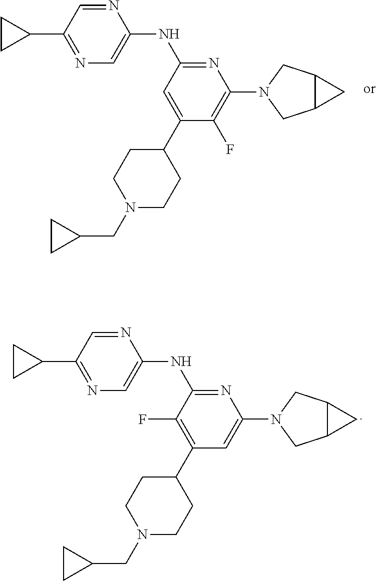 [SMILES: Fc1c(C2CCN(CC3CC3)CC2)cc(Nc2cnc(C3CC3)cn2)nc1N1CC2CC2C1 ; Fc1c(C2CCN(CC3CC3)CC2)cc(N2CC3CC3C2)nc1Nc1cnc(C2CC2)cn1]

In some embodiments, provided herein is a compound of Formula (I), (Ia), or (Ib), or a pharmaceutically acceptable salt or solvate thereof, having the structure:

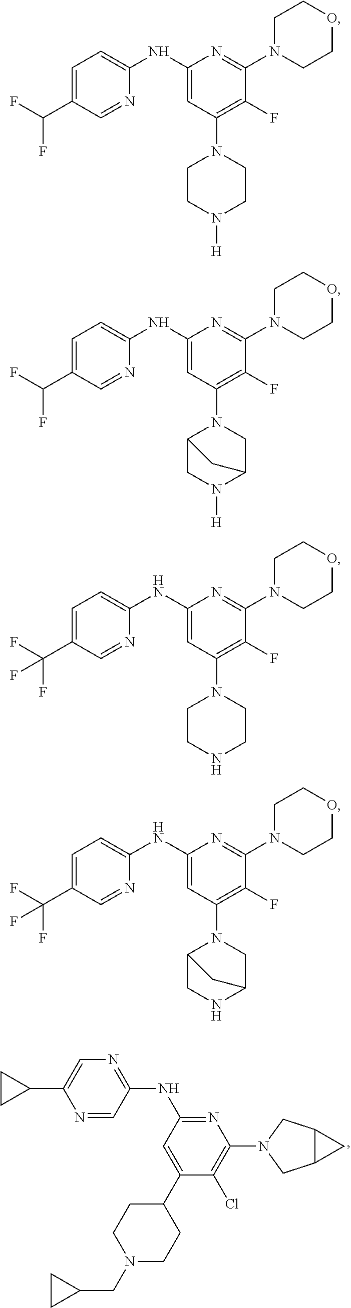 [SMILES: Fc1c(N2CCNCC2)cc(Nc2ccc(C(F)F)cn2)nc1N1CCOCC1 ; Fc1c(N2CC3CC2CN3)cc(Nc2ccc(C(F)F)cn2)nc1N1CCOCC1 ; Fc1c(N2CCNCC2)cc(Nc2ccc(C(F)(F)F)cn2)nc1N1CCOCC1 ; Fc1c(N2CC3CC2CN3)cc(Nc2ccc(C(F)(F)F)cn2)nc1N1CCOCC1 ; Clc1c(C2CCN(CC3CC3)CC2)cc(Nc2cnc(C3CC3)cn2)nc1N1CC2CC2C1]

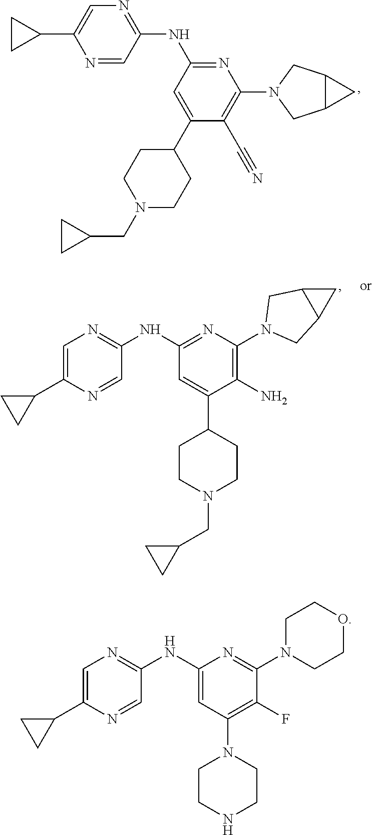 [SMILES: N#Cc1c(C2CCN(CC3CC3)CC2)cc(Nc2cnc(C3CC3)cn2)nc1N1CC2CC2C1 ; Nc1c(C2CCN(CC3CC3)CC2)cc(Nc2cnc(C3CC3)cn2)nc1N1CC2CC2C1 ; Fc1c(N2CCNCC2)cc(Nc2cnc(C3CC3)cn2)nc1N1CCOCC1]

In another aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zurich: Wiley-VCH/VHCA, 2002. Pharmaceutical salts typically are more soluble and more rapidly soluble in stomach and intestinal fluids than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound described herein with an acid to provide a “pharmaceutically acceptable acid addition salt.” In some embodiments, the compound described herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (− L); malonic acid; mandelic acid (DL); methanesulfonic acid; monomethyl fumarate, naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (− L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt.

In some embodiments, pharmaceutically acceptable salts are obtained by reacting a compound described herein with a base to provide a “pharmaceutically acceptable base addition salt.”

In some embodiments, the compound described herein is acidic and is reacted with a base.

In such situations, an acidic proton of the compound described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of isolating or purifying the compound with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), or pharmaceutically acceptable salts of compounds described herein, as well as active metabolites of these compounds having the same type of activity.

In some embodiments, sites on the organic groups (e.g., alkyl groups, aromatic rings) of compounds described herein are susceptible to various metabolic reactions. Incorporation of appropriate substituents on the organic groups will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g., with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, one or more hydrogen atoms of the compounds described herein is replaced with deuterium.

In some embodiments, the compounds described herein possess one or more stereocenters and each stereocenter exists independently in either the R or S configuration. The compounds presented herein include all diastereomeric, enantiomeric, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility.

In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In some embodiments, stereoisomers are obtained by stereoselective synthesis.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

In additional or further embodiments, the compounds are rapidly metabolized in plasma.

In additional or further embodiments, the compounds are rapidly metabolized by the intestines.

In additional or further embodiments, the compounds are rapidly metabolized by the liver.

### Synthesis of Compounds

Compounds described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions. The starting materials are available from commercial sources or are readily prepared.

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

In some embodiments, compounds are prepared as described in the Examples.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ or C₁₋ₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄alkyl” or “C₁₋₄alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, 4 carbon atoms, 5 carbon atoms, 6 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl group. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. In certain embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises two carbon atoms (e.g., C₂ alkylene). In other embodiments, an alkylene comprises two to four carbon atoms (e.g., C₂-C₄ alkylene). Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Deuteroalkyl” refers to an alkyl group where 1 or more hydrogen atoms of an alkyl are replaced with deuterium.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃, —C≡CCH₂CH₃, and —CH₂C≡CH.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

The term “aromatic” refers to a planar ring having a delocalized π-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon or nitrogen atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycle includes cycloalkyl and aryl.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic group, wherein each of the atoms forming the ring (i.e. skeletal atoms) is a carbon atom. In some embodiments, cycloalkyls are spirocyclic or bridged compounds. In some embodiments, cycloalkyls are fully saturated. In some embodiments, cycloalkyls are partially unsaturated. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicyclo[1.1.1]pentyl. In some embodiments, a cycloalkyl is a C₃-C₆cycloalkyl. In some embodiments, a cycloalkyl is a monocyclic cycloalkyl. Monocyclic cycloalkyls include, but are not limited to, cyclopropyl, cyclobutyl, cyclopentyl, cyclohexyl, cycloheptyl, and cyclooctyl. Polycyclic cycloalkyls include, for example, adamantyl, norbornyl (i.e., bicyclo[2.2.1]heptanyl), norbornenyl, decalinyl, 7,7-dimethyl-bicyclo[2.2.1]heptanyl, and the like.

The term “halo” or, alternatively, “halogen” or “halide” means fluoro, chloro, bromo or iodo. In some embodiments, halo is fluoro, chloro, or bromo.

The term “haloalkyl” refers to an alkyl in which one or more hydrogen atoms are replaced by a halogen atom. In one aspect, a fluoroalkyl is a C₁-C₆fluoroalkyl.

The term “fluoroalkyl” refers to an alkyl in which one or more hydrogen atoms are replaced by a fluorine atom. In one aspect, a fluoroalkyl is a C₁-C₆fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “heteroalkyl” refers to an alkyl group in which one or more skeletal atoms of the alkyl are selected from an atom other than carbon, e.g., oxygen, nitrogen (e.g., —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “heteroalkylene” refers to a divalent heteroalkyl group.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, benzotriazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, heterocycloalkyls are spirocyclic or bridged compounds. In some embodiments, heterocycloalkyls are fully saturated. In some embodiments, heterocycloalkyls are partially unsaturated. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-Cioheterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s). In some other embodiments, optional substituents are individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, —CH₂CO₂H, —CH₂CO₂alkyl, —CH₂C(═O)NH₂, —CH₂C(═O)NH(alkyl), —CH₂C(═O)N(alkyl)₂, —CH₂S(═O)₂NH₂, —CH₂S(═O)₂NH(alkyl), —CH₂S(═O)₂N(alkyl)₂, alkyl, alkenyl, alkynyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, substituted groups are substituted with one of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may also be formulated in rectal compositions such as suppositories or retention enemas, e.g., containing conventional suppository bases such as cocoa butter, polyethylene glycol, or other glycerides.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

In some embodiments, a compound disclosed herein is formulated to provide a controlled release of the compound. Controlled release refers to the release of the compound described herein from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

Approaches to deliver the intact therapeutic compound to the particular regions of the gastrointestinal tract (e.g., such as the colon), include:
(i) Coating with polymers: The intact molecule can be delivered to the colon without absorbing at the upper part of the intestine by coating of the drug molecule with the suitable polymers, which degrade only in the colon.
(ii) Coating with pH-sensitive polymers: The majority of enteric and colon targeted delivery systems are based on the coating of tablets or pellets, which are filled into conventional hard gelatin capsules. Most commonly used pH-dependent coating polymers are methacrylic acid copolymers, commonly known as Eudragit® S, more specifically Eudragit® L and Eudragit® S. Eudragit® L100 and S 100 are copolymers of methacrylic acid and methyl methacrylate. Additional pH-dependent coating polymers include cellulose acetate phthalate (CAP), hydroxypropyl methylcellulose phthalate (HPMCP), polyvinyl acetate phthalate (PVAP) and cellulose acetate trimelliate.
(iii) Coating with biodegradable polymers;
(iv) Embedding in matrices;
(v) Embedding in biodegradable matrices and hydrogels;
(vi) Embedding in pH-sensitive matrices;
(vii) Timed release systems;
(viii) Redox-sensitive polymers;
(ix) Bioadhesive systems;
(x) Coating with microparticles;
(xi) Osmotic controlled drug delivery.

Another approach towards colon-targeted drug delivery or controlled-release systems includes embedding the drug in polymer matrices to trap it and release it in the colon. These matrices can be pH-sensitive or biodegradable. Matrix-Based Systems, such as multi-matrix (MMX)-based delayed-release tablets, ensure the drug release in the colon.

Additional pharmaceutical approaches to targeted delivery of therapeutics to particular regions of the gastrointestinal tract are known. Chourasia M K, Jain S K, Pharmaceutical approaches to colon targeted drug delivery systems., J Pharm Sci. 2003 Jan.-Apr. 6(1):33-66. Patel M, Shah T, Amin A. Therapeutic opportunities in colon-specific drug-delivery systems Crit Rev Ther Drug Carrier Syst. 2007; 24(2):147-202. Kumar P, Mishra B. Colon targeted drug delivery systems—an overview. Curr Drug Deliv. 2008 July; 5(3):186-98. Van den Mooter G. Colon drug delivery. Expert Opin Drug Deliv. 2006 January; 3(1):111-25. Seth Amidon, Jack E. Brown, and Vivek S. Dave, Colon-Targeted Oral Drug Delivery Systems: Design Trends and Approaches, AAPS PharmSciTech. 2015 August; 16(4): 731-741.

It should be understood that in addition to the ingredients particularly mentioned above, the compounds and compositions described herein may include other agents conventional in the art having regard to the type of formulation in question, for example those suitable for oral administration may include flavoring agents.

### Methods of Dosing and Treatment Regimens

In one embodiment, the compounds described herein, or a pharmaceutically acceptable salt thereof, are used in the preparation of medicaments for the treatment of diseases or conditions in a mammal that would benefit from administration of an LZK inhibitor. Methods for treating any of the diseases or conditions described herein in a mammal in need of such treatment, involves administration of pharmaceutical compositions that include at least one compound described herein or a pharmaceutically acceptable salt or solvate thereof, in therapeutically effective amounts to said mammal.

In some embodiments, described herein is a method of treating cancer in a mammal, comprising administering to the mammal a compound of Formula (I), (Ia), or (Ib), or a pharmaceutically acceptable salt or solvate thereof. In some embodiments, described herein is a method of treating cancer in a mammal, comprising administering to the mammal a compound of Formula (I), (Ia), or (Ib), or a pharmaceutically acceptable salt or solvate thereof, wherein the cancer is a cancer with LZK alterations. In some embodiments, the cancer is a squamous cell carcinoma. In some embodiments, the cancer is head and neck squamous cell carcinoma. In some embodiments, the cancer is esophageal squamous cell carcinoma.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder, or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

### Examples

The following examples are provided for illustrative purposes only and not to limit the scope of the claims provided herein.

As used above, and throughout the description of the invention, the following abbreviations, unless otherwise indicated, shall be understood to have the following meanings:
acac acetylacetone
ACN or MeCN acetonitrile
AcOH acetic acid
Ac acetyl
BINAP 2,2′-bis(diphenylphosphino)-1,1′-binaphthalene
Bn benzyl
BOC or Boc tert-butyl carbamate
i-Bu iso-butyl
t-Bu tert-butyl
Cy cyclohexyl
CDI 1,1-carbonyldiimidazole
CPME cyclopentyl methyl ether
DAST (diethylamino)sulfur trifluoride
DBA or dba dibenzylideneacetone
DCE dichloroethane (ClCH₂CH₂C₁)
DCM dichloromethane (CH₂Cl₂)
DHP 3,4-dihydropyran
DIBAL-H diisobutylaluminum hydride
DIPEA or DIEA diisopropylethylamine
DMAP 4-(N,N-dimethylamino)pyridine
DME 1,2-dimethoxyethane
DMF N,N-dimethylformamide
DMA N,N-dimethylacetamide
DMPU N,N-dimethylpropyleneurea
DMSO dimethylsulfoxide
Dppf or dppf 1,1′-bis(diphenylphosphino)ferrocene
EDC or EDCI N-(3-dimethylaminopropyl)-N′-ethylcarbodiimide hydrochloride
EEDQ 2-ethoxy-1-ethoxycarbonyl-1,2-dihydroquinoline
eq equivalent(s)
Et ethyl
Et₂O diethyl ether
EtOH ethanol
EtOAc ethyl acetate
HATU 1-[bis(dimethylamino)methylene]-1H-1,2,3-triazolo[4,5-b]pyridinium 3-oxid hexafluorophosphate
HMPA hexamethylphosphoramide
HOBt 1-hydroxybenzotriazole
HPLC high performance liquid chromatography
IBX 2-iodoxybenzoic acid
KOAc potassium acetate
KHMDS potassium bis(trimethylsilyl)amide
NaHMDS sodium bis(trimethylsilyl)amide
LiHMDS lithium bis(trimethylsilyl)amide
LAH lithium aluminum anhydride
LCMS liquid chromatography mass spectrometry
2-MeTHF 2-methyltetrahydrofuran
Me methyl
MeOH methanol
MOM methoxymethyl ether
MS mass spectroscopy
Ms mesyl
MTBE methyl tert-butyl ether
NBS N-bromosuccinimide
NCS N-chlorosuccinimide
NIS N-iodosuccinimide
NMI 1-methylimidazole
NMM N-methyl-morpholine
NMP N-methyl-pyrrolidin-2-one
NMR nuclear magnetic resonance
OTf trifluoromethanesulfonate
PCC pyridinium chlorochromate
PE petroleum ether
PG protecting group
Ph phenyl
PPTS pyridium p-toluenesulfonate
iPr/i-Pr iso-propyl
RP-HPLC reverse-phase high-pressure liquid chromatography
rt room temperature
SEM 2-(trimethylsilyl)ethoxymethyl
TBS tert-butyldimethylsilyl
TBAF tetra-n-butylammonium fluoride
TBAI tetra-n-butylammonium iodide
TCFH chloro-N,N,N′,N′-tetramethylformamidinium hexafluorophosphate
TEA triethylamine
TFA trifluoroacetic acid
THF tetrahydrofuran
TLC thin layer chromatography
TMEDA N,N,N′,′N-tetramethylethylenediamine
TMS trimethylsilyl
TsOH/p-TsOH p-toluenesulfonic acid

### Example 1: N-(6-(3-Azabicyclo[3.1.0]hexan-3-yl)-4-(1-(cyclopropylmethyl)piperidin-4-yl)-5-fluoropyridin-2-yl)-5-cyclopropylpyrazin-2-amine (Compound 1)

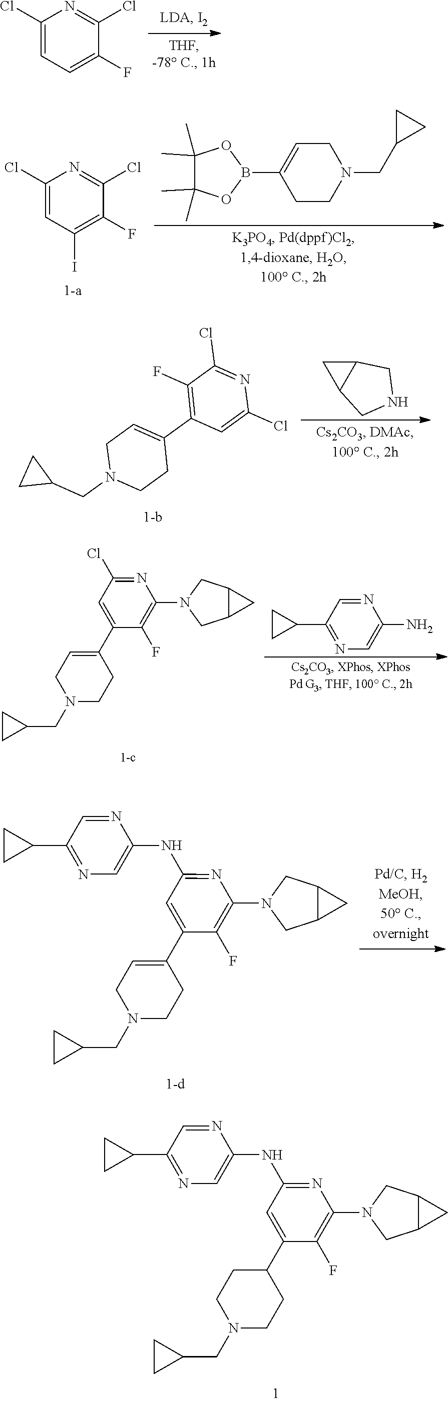

### Step 1: 2,6-Dichloro-3-fluoro-4iodopyridine

To a stirred solution of 2,6-dichloro-3-fluoropyridine (3 g, 18.07 mmol, 1 equiv) in tetrahydrofuran (15 mL) was added LDA (10 mL, 19.88 mmol, 1.1 equiv, 2M in THF) dropwise at −78° C. under nitrogen atmosphere. The resulting mixture was stirred for 1 h at −78° C. under nitrogen atmosphere. To the above mixture was added a solution of I₂ (5.96 g, 23.5 mmol, 1.3 equiv) dropwise in tetrahydrofuran (15 mL) at −78° C. The resulting mixture was stirred for additional 1 h at −78° C. The reaction was quenched by the addition of sat. NH₄Cl (aq.) (40 mL) at room temperature. The resulting mixture was extracted with EtOAc (3×100 mL). The combined organic layers were washed with saturated sodium thiosulfate solution (1×100 mL) and brine (2×100 mL), dried over anhydrous Na₂SO₄, filtered, and concentrated under reduced pressure. The residue was purified by silica gel column chromatography to afford 2,6-dichloro-3-fluoro-4iodopyridine (4.78 g, 91%) as a yellow solid. LC-MS: (ES, m/z): [M+H]⁺=291.9.

### Step 2: 2′,6′-Dichloro-1-(cyclopropylmethyl)-3′-fluoro-1,2,3,6-tetrahydro-4,4′-bipyridine

To a stirred solution of 2,6-dichloro-3-fluoro-4iodopyridine (300 mg, 1.03 mmol, 1 equiv) and 1-(cyclopropylmethyl)-4-(4,4,5,5-tetramethyl-1,3,2-dioxaborolan-2-yl)-3,6-dihydro-2H-pyridine (298 mg, 1.13 mmol, 1.1 equiv) and K₃PO₄ (545 mg, 2.57 mmol, 2.5 equiv) in 1,4-dioxane (6 mL) and H₂O (1.2 mL) was added Pd(dppf)Cl₂ (75.2 mg, 0.10 mmol, 0.1 equiv) at room temperature under nitrogen atmosphere. The resulting mixture was stirred for 2 h at 100° C. under nitrogen atmosphere. The mixture was cooled to room temperature and quenched by the addition of water (10 mL). The resulting mixture was extracted with EtOAc (3×20 mL). The combined organic layers were washed with brine (1×40 mL), dried over anhydrous Na₂SO₄, filtered, and concentrated under reduced pressure. The residue was purified by reversed-phase flash chromatography with the following conditions: column, C18 silica gel; mobile phase, MeCN in water (10 mmol/L NH₄HCO₃), 0% to 100% gradient in 10 min; detector, UV 254 nm to afford 2′,6′-dichloro-1-(cyclopropylmethyl)-3′-fluoro-1,2,3,6-tetrahydro-4,4′-bipyridine (290 mg, 94%) as a black oil. LC-MS: (ES, m/z): [M+H]⁺=301.0.

### Step 3: 3-(6-Chloro-1′-(cyclopropylmethyl)-3-fluoro-1′,2′,3′,6′-tetrahydro-[4,4′-bipyridin]-2-yl)-3-azabicyclo[3.1.0]hexane

To a stirred solution of 2′,6′-dichloro-1-(cyclopropylmethyl)-3′-fluoro-1,2,3,6-tetrahydro-4,4′-bipyridine (340 mg, 1.13 mmol, 1 equiv) and 3-azabicyclo[3.1.0]hexane (113 mg, 1.36 mmol, 1.2 equiv) in DMA (6.8 mL) was added Cs₂CO₃ (736 mg, 2.26 mmol, 2 equiv) at room temperature under nitrogen atmosphere. The resulting mixture was stirred for 2 h at 100° C. under nitrogen atmosphere. The mixture was cooled to room temperature and quenched by the addition of water (10 mL). The resulting mixture was extracted with EtOAc (3×20 mL). The combined organic layers were washed with brine (1×30 mL), dried over anhydrous Na₂SO₄, filtered, and concentrated under reduced pressure. The residue was purified by reversed-phase flash chromatography with the following conditions: column, C18 silica gel; mobile phase, MeCN in water (0.1% FA), 0% to 100% gradient in 10 min; detector, UV 254 nm to afford 3-(6-chloro-1′-(cyclopropylmethyl)-3-fluoro-1′,2′,3′,6′-tetrahydro-[4,4′-bipyridin]-2-yl)-3-azabicyclo[3.1.0]hexane (76 mg, 19%) as a black oil. LC-MS: (ES, m/z): [M+H]⁺=348.3.

### Step 4: 6-(3-Azabicyclo[3.1.0]hexan-3-yl)-1′-(cyclopropylmethyl)-N-(5-cyclopropylpyrazin-2-yl)-5-fluoro-1′,2′,3′,6′-tetrahydro-[4,4′-bipyridin]-2-amine

To a stirred solution of 3-(6-chloro-1′-(cyclopropylmethyl)-3-fluoro-1′,2′,3′,6′-tetrahydro-[4,4′-bipyridin]-2-yl)-3-azabicyclo[3.1.0]hexane (76 mg, 0.22 mmol, 1 equiv) and 5 -cyclopropylpyrazin-2-amine (35 mg, 0.26 mmol, 1.2 equiv) and Cs₂CO₃ (214 mg, 0.65 mmol, 3 equiv) in THE (1.5 mL) was added XPhos (15 mg, 0.032 mmol, 0.15 equiv) and XPhos Pd G3 (15 mg, 0.02 mmol, 0.08 equiv) at room temperature under nitrogen atmosphere. The resulting mixture was stirred for 2 h at 100° C. under nitrogen atmosphere. The mixture was cooled to room temperature. The resulting mixture was filtered and the filter cake was washed with tetrahydrofuran (2×1 mL). The filtrate was concentrated under reduced pressure. The residue was purified by silica gel column chromatography to afford 6-(3-azabicyclo[3.1.0]hexan-3-yl)-1′-(cyclopropylmethyl)-N-(5-cyclopropylpyrazin-2-yl)-5-fluoro-1′,2′,3′,6′-tetrahydro-[4,4′-bipyridin]-2-amine (75 mg, 77%) as a yellow oil. LC-MS (ES, m/z): [M+H]⁺=447.3.

### Step 5: N-(6-(3-Azabicyclo[3.1.0]hexan-3-yl)-4-(1-(cyclopropylmethyl)piperidin-4-yl)-5-fluoropyridin-2-yl)-5-cyclopropylpyrazin-2-amine (Compound 1)

To a stirred solution of 6-(3-azabicyclo[3.1.0]hexan-3-yl)-1′-(cyclopropylmethyl)-N-(5-cyclopropylpyrazin-2-yl)-5-fluoro-1′,2′,3′,6′-tetrahydro-[4,4′-bipyridin]-2-amine (75 mg, 0.17 mmol, 1 equiv) in MeOH (4 mL) was added Pd/C (75 mg, 10% wt) at room temperature. The resulting mixture was stirred for overnight at 50° C. under hydrogen atmosphere. The mixture cooled to room temperature, filtered, and concentrated. The crude product (75 mg) was purified by Prep-HPLC with the following conditions (Column: XBridge Prep OBD C18 Column 30*150 mm, 5 m; Mobile Phase A: water (10 mmol/L NH₄HCO₃), Mobile Phase B: ACN; Flow rate: 60 mL/min mL/min; Gradient: 55% B to 71% B in 10 min; Wave Length: 254 nm/220 nm nm) to afford N-(6-(3-azabicyclo[3.1.0]hexan-3-yl)-4-(1-(cyclopropylmethyl)piperidin-4-yl)-5-fluoropyridin-2-yl)-5-cyclopropylpyrazin-2-amine (Compound 1) (9 mg, 11%) as a white solid. LC-MS: (ES, m/z): [M+H]⁺=449.3. ¹H-NMR (400 MHz, DMSO-d₆): δ 9.34 (s, 1H), 9.03 (d, J=1.5 Hz, 1H), 8.16 (d, J=1.5 Hz, 1H), 6.61 (d, J=3.2 Hz, 1H), 3.81 (dd, J=10.2, 3.2 Hz, 2H), 3.51 (d, J=10.0 Hz, 2H), 3.06 (dt, J=11.6, 3.2 Hz, 2H), 2.71-2.61 (m, 1H), 2.19 (d, J=6.5 Hz, 2H), 2.13-1.91 (m, 3H), 1.79-1.47 (m, 6H), 0.96-0.88 (m, 2H), 0.84 (tt, J=4.5, 2.5 Hz, 3H), 0.67 (td, J=7.7, 4.4 Hz, 1H), 0.54-0.37 (m, 2H), 0.26 (q, J=4.1 Hz, 1H), 0.11-0.03 (m, 2H). ¹⁹F-NMR (376 MHz, DMSO-d₆): δ −153.73.

### Example 2: N-(6-(3-Azabicyclo[3.1.0]hexan-3-yl)-4-(1-(cyclopropylmethyl)piperidin-4-yl)-3-fluoropyridin-2-yl)-5-cyclopropylpyrazin-2-amine (Compound 2)

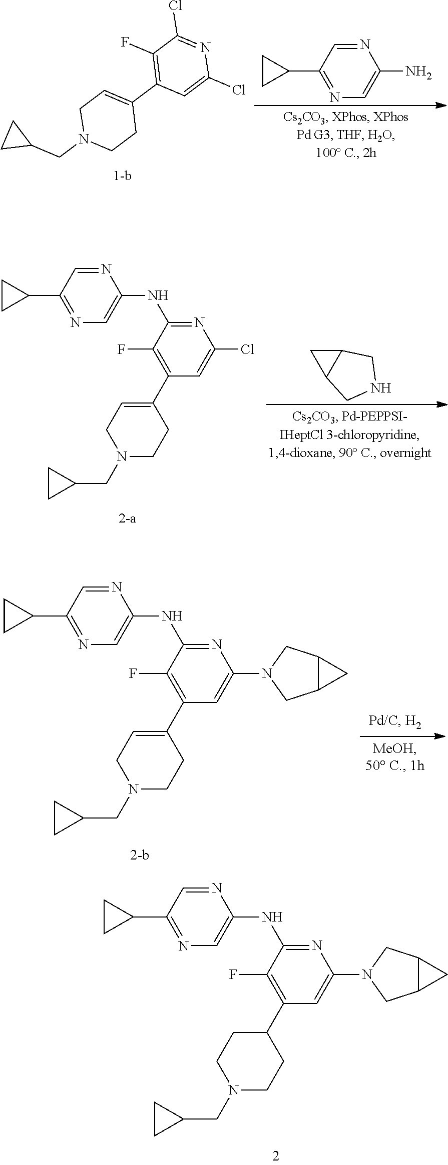

Compound 1-b was synthesized as described in Example 1.

### Step 1: 6-Chloro-1′-(cyclopropylmethyl)-N-(5-cyclopropylpyrazin-2-yl)-3-fluoro-1′,2′,3′,6′-tetrahydro-[4,4′-bipyridin]-2-amine

To a stirred solution of 2′,6′-dichloro-1-(cyclopropylmethyl)-3′-fluoro-3,6-dihydro-2H-4,4′-bipyridine (775 mg, 2.57 mmol, 1.5 equiv) and 5-cyclopropylpyrazin-2-amine (232 mg, 1.72 mmol, 1 equiv) and Cs₂CO₃ (1.68 g, 5.15 mmol, 3 equiv) in THF (6 mL) was added XPhos (46 mg, 0.10 mmol, 0.06 equiv) and XPhos Pd G3 (46 mg, 0.055 mmol, 0.03 equiv) at room temperature under nitrogen atmosphere. The resulting mixture was stirred for 2 h at 100° C. under nitrogen atmosphere. The mixture was cooled to room temperature and quenched by the addition of water (10 mL). The resulting mixture was extracted with EtOAc (3×20 mL). The combined organic layers were washed with brine (2×40 mL), dried over anhydrous Na₂SO₄, filtered, and concentrated under reduced pressure. The residue was purified by reversed-phase flash chromatography with the following conditions: column, C18 silica gel; mobile phase, MeCN in water (0.1% FA), 0% to 100% gradient in 10 min; detector, UV 254 nm to afford 6-chloro-1′-(cyclopropylmethyl)-N-(5-cyclopropylpyrazin-2-yl)-3-fluoro-1′,2′,3′,6′-tetrahydro-[4,4′-bipyridin]-2-amine (378 mg, 55%) as a yellow oil. LC-MS: (ES, m/z): [M+H]⁺=400.3.

### Step 2: 6-(3-Azabicyclo[3.1.0]hexan-3-yl)-1′-(cyclopropylmethyl)-N-(5-cyclopropylpyrazin-2-yl)-3-fluoro-1′,2′,3′,6′-tetrahydro-[4,4′-bipyridin]-2-amine

To a stirred solution of 6-chloro-1′-(cyclopropylmethyl)-N-(5-cyclopropylpyrazin-2-yl)-3-fluoro-1′,2′,3′,6′-tetrahydro-[4,4′-bipyridin]-2-amine (200 mg, 0.50 mmol, 1 equiv) and 3-azabicyclo[3.1.0]hexane (50 mg, 0.60 mmol, 1.2 equiv) and Cs₂CO₃ (489 mg, 1.50 mmol, 3 equiv) in 1,4-dioxane (4 mL) was added {1,3-bis[2,6-bis(heptan-4-yl)phenyl]-4,5-dichloro-2,3-dihydro-1H-imidazol-2-yl}dichloro(3-chloro-1lambda4-pyridin-1-yl)palladium (49 mg, 0.05 mmol, 0.1 equiv) at room temperature under nitrogen atmosphere. The resulting mixture was stirred for overnight at 90° C. under nitrogen atmosphere. The mixture was cooled to room temperature and quenched by the addition of water (5 mL). The resulting mixture was extracted with EtOAc (3×10 mL). The combined organic layers were washed with brine (1×20 mL), dried over anhydrous Na₂SO₄, filtered, and concentrated under reduced pressure. The residue was purified by reversed-phase flash chromatography with the following conditions: column, C18 silica gel; mobile phase, MeCN in water (0.1% TFA), 0% to 100% gradient in 10 min; detector, UV 254 nm to afford 6-(3-azabicyclo[3.1.0]hexan-3-yl)-1′-(cyclopropylmethyl)-N-(5-cyclopropylpyrazin-2-yl)-3-fluoro-1′,2′,3′,6′-tetrahydro-[4,4′-bipyridin]-2-amine (120 mg, 54%) as a yellow oil. LC-MS: (ES, m/z): [M+H]⁺=447.3.

### Step 3: N-(6-{3-Azabicyclo[3.1.0]hexan-3-yl}-4-[1-(cyclopropylmethyl)piperidin-4-yl]-3-fluoropyridin-2-yl)-5-cyclopropylpyrazin-2-amine (Compound 2)

To a stirred solution of 6-(3-azabicyclo[3.1.0]hexan-3-yl)-1′-(cyclopropylmethyl)-N-(5-cyclopropylpyrazin-2-yl)-3-fluoro-1′,2′,3′,6′-tetrahydro-[4,4′-bipyridin]-2-amine (10 mg, 0.02 mmol, 1 equiv) in MeOH (2 mL) was added Pd/C (10%, 10 mg, 0.09 mmol, 4.2 equiv) at room temperature under air atmosphere. The resulting mixture was stirred for 1 h at 50° C. under hydrogen atmosphere. The mixture was cooled to room temperature, filtered, and concentrated. The residue was purified by Prep-HPLC with the following conditions (Column: Sunfire prep C18 column, 30*150 mm, 5 m; Mobile Phase A: Water (0.1% FA), Mobile Phase B: MeOH—HPLC; Flow rate: 60 mL/min mL/min; Gradient: isocratic 27% B t45% B in 10 min; Wave Length: 254 nm/220 nm) to afford N-(6-(3-azabicyclo[3.1.0]hexan-3-yl)-4-(1-(cyclopropylmethyl)piperidin-4-yl)-3-fluoropyridin-2-yl)-5-cyclopropylpyrazin-2-amine (Compound 2) (2 mg, 15%) as a white solid. LC-MS: (ES, m/z): [M+H]=449.3. ¹H-NMR (400 MHz, DMSO-d₆): δ 9.28 (s, 1H), 8.80 (s, 1H), 8.24 (s, 1H), 5.86 (d, J=3.0 Hz, 1H), 3.62 (d, J=9.9 Hz, 2H), 3.12 (d, J=11.1 Hz, 3H), 2.77-2.67 (m, 1H), 2.24 (d, J=6.5 Hz, 2H), 2.05-2.14 (m, 1H), 2.04 (t, J=10.8 Hz, 2H), 1.83-1.69 (m, 6H), 0.99 (dd, J=9.0, 6.0 Hz, 2H), 0.90 (t, J=7.9 Hz, 4H), 0.75 (td, J=7.8, 4.3 Hz, 1H), 0.50 (d, J=7.7 Hz, 2H), 0.24 (dd, J=4.1 Hz, 1H), 0.12 (d, J=5.0 Hz, 2H). ¹⁹F-NMR (377 MHz, DMSO-d₆): δ −159.26.

### Example 3: N-(5-(Difluoromethyl)pyridin-2-yl)-5-fluoro-6-morpholino-4-(piperazin-1-yl)pyridin-2-amine di-TFA salt (Compound 3)

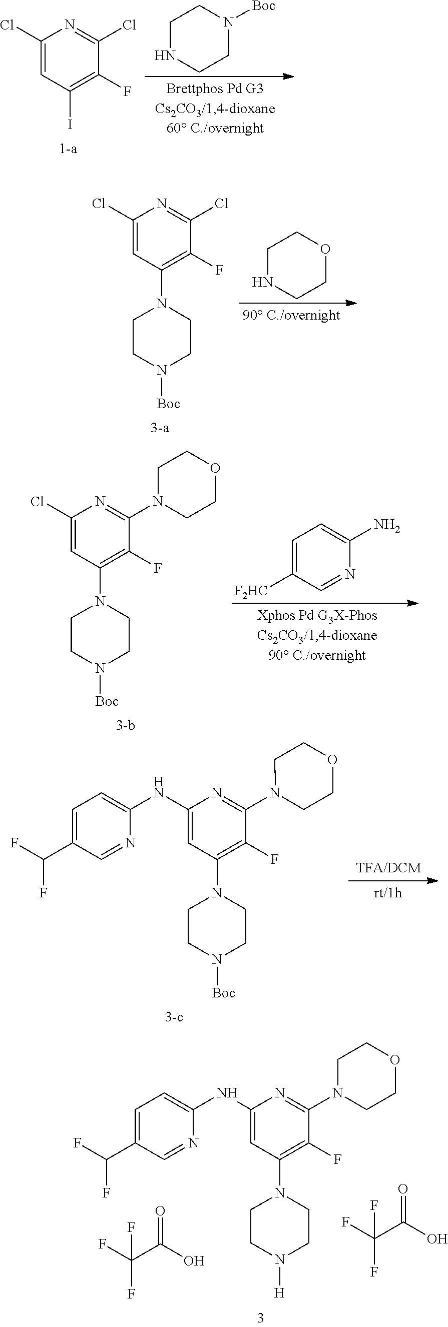

Compound 1-a was synthesized as described in Example 1.

### Step 1: tert-Butyl 4-(2,6-dichloro-3-fluoropyridin-4-yl)piperazine-1-carboxylate

To a stirred solution of 2,6-dichloro-3-fluoro-4iodopyridine (3.5 g, 11.992 mmol, 1 equiv) and tert-butyl piperazine-1-carboxylate (2.68 g, 14.39 mmol, 1.2 equiv) in 1,4-dioxane (30 mL) were added Cs₂CO₃ (7.81 g, 23.984 mmol, 2 equiv) and BrettPhos Pd G3 (1.09 g, 1.199 mmol, 0.1 equiv). The resulting mixture was stirred overnight at 60° C. under nitrogen atmosphere. The reaction mixture was cooled to room temperature and quenched with water (100 mL). The resulting mixture was extracted with EtOAc (3×500 mL). The combined organic layers were washed with brine (1×100 mL) and dried over anhydrous Na₂SO₄. After filtration, the filtrate was concentrated under reduced pressure. The residue was purified by silica gel column chromatography to afford tert-butyl 4-(2,6-dichloro-3-fluoropyridin-4-yl)piperazine-1-carboxylate (460 mg, 11%) as a brown solid. LC-MS: (ES, m/z): [M+H]⁺=349.1.

### Step 2: tert-Butyl 4-(6-chloro-3-fluoro-2-morpholinopyridin-4-yl)piperazine-1-carboxylate

A mixture of tert-butyl 4-(2,6-dichloro-3-fluoropyridin-4-yl)piperazine-1-carboxylate (330 mg, 0.942 mmol, 1 equiv) and morpholine (123 mg, 1.413 mmol, 1.5 equiv) was stirred overnight at 90° C. The reaction mixture was cooled to room temperature and quenched with water (50 mL). The resulting mixture was extracted with EtOAc (3×100 mL). The combined organic layers were washed with brine (1×50 mL) and dried over anhydrous Na₂SO₄. After filtration, the filtrate was concentrated under reduced pressure. The residue was purified by silica gel column chromatography to afford tert-butyl 4-[6-chloro-3-fluoro-2-(morpholin-4-yl)pyridin-4-yl]piperazine-1-carboxylate (300 mg, 79%) as a brown solid. LC-MS: (ES, m/z): [M+H]=400.2.

### Step 3: tert-Butyl 4-(6-{[5-(difluoromethyl)pyridin-2-yl]amino}-3-fluoro-2-(morpholin-4-yl)pyridin-4-yl)piperazine-1-carboxylate

To a stirred solution of tert-butyl 4-[6-chloro-3-fluoro-2-(morpholin-4-yl)pyridin-4-yl]piperazine-1-carboxylate (100 mg, 0.249 mmol, 1 equiv) and 5-(difluoromethyl)pyridin-2-amine (54 mg, 0.373 mmol, 1.5 equiv) in 1,4-dioxane (5 mL) were added Cs₂CO₃ (163 mg, 0.498 mmol, 2 equiv), X-Phos (12 mg, 0.025 mmol, 0.1 equiv) and 3rd Generation XPhos precatalyst (21 mg, 0.025 mmol, 0.1 equiv). The resulting mixture was stirred overnight at 90° C. under nitrogen atmosphere. The reaction mixture was cooled to room temperature and quenched with water (50 mL). The resulting mixture was extracted with EtOAc (3×100 mL). The combined organic layers were washed with brine (1×20 mL) and dried over anhydrous Na₂SO₄. After filtration, the filtrate was concentrated under reduced pressure. The residue was purified by silica gel column chromatography to afford tert-butyl 4-(6-{[5-(difluoromethyl)pyridin-2-yl]amino}-3-fluoro-2-(morpholin-4-yl)pyridin-4-yl)piperazine-1-carboxylate (120 mg, 95%) as a brown solid. LC-MS: (ES, m/z): [M+H]⁺=508.2.

### Step 4: N-(5-(Difluoromethyl)pyridin-2-yl)-5-fluoro-6-morpholino-4-(piperazin-1-yl)pyridin-2-amine di-TFA salt (Compound 3)

To a stirred solution of tert-butyl 4-(6-{[5-(difluoromethyl)pyridin-2-yl]amino}-3-fluoro-2-(morpholin-4-yl)pyridin-4-yl)piperazine-1-carboxylate (120 mg, 0.236 mmol, 1 equiv) in DCM (5 mL) was added TFA (5 mL). The resulting mixture was stirred for 1 h at room temperature. The resulting mixture was concentrated under vacuum. The crude product (150 mg) was purified by Prep-HPLC with the following conditions (Column: Sunfire prep C18 column, 30*150 mm, 5 m; Mobile Phase A: Water (0.05% TFA), Mobile Phase B: ACN; Flow rate: 60 mL/min; Gradient: isocratic 2% B to 18% B in 10 min; Wave Length: 254 nm/220 nm) to afford N-(5-(difluoromethyl)pyridin-2-yl)-5-fluoro-6-morpholino-4-(piperazin-1-yl)pyridin-2-amine di-TFA salt (Compound 3) (21.5 mg, 22%) as a yellow solid. LC-MS: (ES, m/z): [M+H]⁺=409. ¹H-NMR (400 MHz, DMSO-d₆): δ 10.32 (s, 1H), 9.16 (s, 2H), 8.47 (s, 1H), 7.96-7.94 (m, 1H), 7.79 (d, J=8.4 Hz, 1H), 7.17-6.82 (m, 2H), 3.76 (t, J=4.4 Hz, 4H), 3.41-3.34 (m, 8H), 3.30 (s, 4H). ¹⁹F-NMR (400 MHz, DMSO-d₆): δ −74.72, −108.99.

### Example 4: 4-(2,5-Diazabicyclo[2.2.1]heptan-2-yl)-N-(5-(difluoromethyl)pyridin-2-yl)-5-fluoro-6-morpholinopyridin-2-amine di-TFA salt (Compound 4)

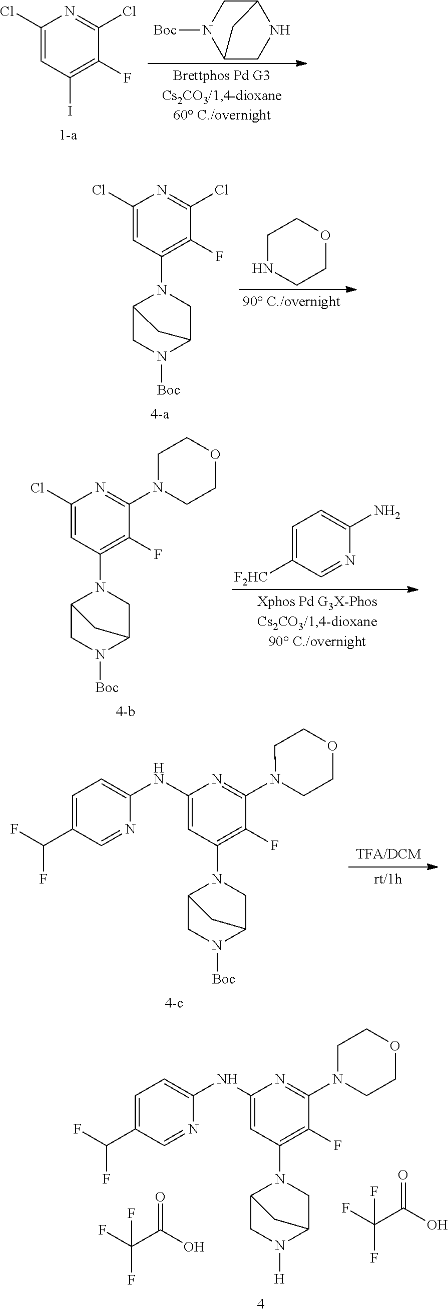

4-(2,5-Diazabicyclo[2.2.1]heptan-2-yl)-N-(5-(difluoromethyl)pyridin-2-yl)-5-fluoro-6-morpholinopyridin-2-amine di-TFA salt (Compound 4) was prepared as described in Example 3 using tert-butyl 2,5-diazabicyclo[2.2.1]heptane-2-carboxylate in Step 1. LC-MS: (ES, m/z): [M+H]⁺=421. ¹H-NMR (400 MHz, DMSO-d₆): δ 9.77 (s, 1H), 9.20 (s, 1H), 8.64 (s, 1H), 8.38 (s, 1H), 7.85 (d, J=9.2 Hz, 1H), 7.77 (d, J=8.8 Hz, 1H), 7.14-6.86 (t, J_{1/2}=J₂₋₃=55.6 Hz, 1H), 6.62 (d, J=4.8 Hz, 1H), 4.61 (s, 1H), 4.44 (s, 1H), 3.77-3.70 (m, 5H), 3.48 (d, J=8.0 Hz, 1H), 3.39-3.23 (m, 6H), 2.16 (d, J=10.8 Hz, 1H), 1.93 (d, J=10.8 Hz, 1H).

### Example 5: 5-Fluoro-6-morpholino-4-(piperazin-1-yl)-N-(5-(trifluoromethyl)pyridin-2-yl)pyridin-2-amine (Compound 5)

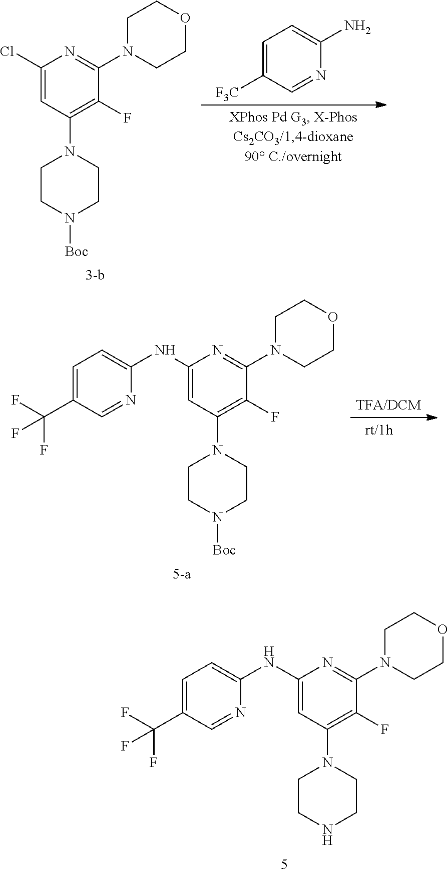 [SMILES: CC(C)(C)OC(=O)N1CCN(c2cc(Cl)nc(N3CCOCC3)c2F)CC1 ; Nc1ccc(C(F)(F)F)cn1 ; CC(C)(C)OC(=O)N1CCN(c2cc(Nc3ccc(C(F)(F)F)cn3)nc(N3CCOCC3)c2F)CC1 ; Fc1c(N2CCNCC2)cc(Nc2ccc(C(F)(F)F)cn2)nc1N1CCOCC1]

5-Fluoro-6-morpholino-4-(piperazin-1-yl)-N-(5-(trifluoromethyl)pyridin-2-yl)pyridin-2-amine (Compound 5) was prepared as described in Example 3 (Steps 3 and 4) using 5-(trifluoromethyl)pyridin-2-amine. LC-MS: (ES, m/z): [M+H]⁺=426.2. ¹H-NMR (400 MHz, DMSO-d₆): δ 9.79 (s, 1H), 8.52 (s, 1H), 7.95 (s, 2H), 6.81 (d, J=4.8 Hz, 1H), 3.73 (t, J=4.4 Hz, 4H), 3.30 (t, J=4.4 Hz, 4H), 3.05 (t, J=3.6 Hz, 4H), 2.83 (t, J=4.8 Hz, 4H).

### Example 6: 4-(2,5-Diazabicyclo[2.2.1]heptan-2-yl)-5-fluoro-6-morpholino-N-(5-(trifluoromethyl)pyridin-2-yl)pyridin-2-amine (Compound 6)

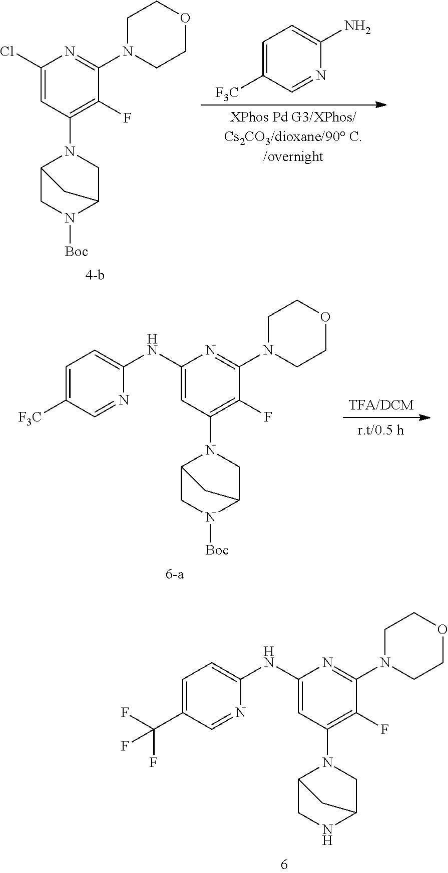 [SMILES: CC(C)(C)OC(=O)N1CC2CC1CN2c1cc(Cl)nc(N2CCOCC2)c1F ; Nc1ccc(C(F)(F)F)cn1 ; CC(C)(C)OC(=O)N1CC2CC1CN2c1cc(Nc2ccc(C(F)(F)F)cn2)nc(N2CCOCC2)c1F ; Fc1c(N2CC3CC2CN3)cc(Nc2ccc(C(F)(F)F)cn2)nc1N1CCOCC1]

4-(2,5-Diazabicyclo[2.2.1]heptan-2-yl)-5-fluoro-6-morpholino-N-(5-(trifluoromethyl)pyridin-2-yl)pyridin-2-amine (Compound 6) was prepared as described in Example 4 using 5-(trifluoromethyl)pyridin-2-amine. LC-MS: (ES, m/z): [M+H]⁺=439. ¹H-NMR (400 MHz, DMSO-d₆): δ 9.61 (s, 1H), 8.48 (s, 1H), 7.97-7.90 (m, 2H), 6.53 (s, 1H), 4.33 (s, 1H), 3.76-3.67 (m, 4H), 3.60-3.58 (m, 2H), 3.29 (d, J=4.8 Hz, 2H), 3.29-3.19 (m, 4H), 3.11 (d, J=8.0 Hz, 1H), 2.89 (dd, J₁₋₂=9.6 Hz, J₁₋₃=22.8 Hz, 2H), 1.77 (d, J=8.8 Hz, 1H), 1.65 (d, J=8.8 Hz, 1H).

### Example 7: N-(6-(3-Azabicyclo[3.1.0]hexan-3-yl)-5-chloro-4-(1-(cyclopropylmethyl)piperidin-4-yl)pyridin-2-yl)-5-cyclopropylpyrazin-2-amine TFA salt (Compound 7)

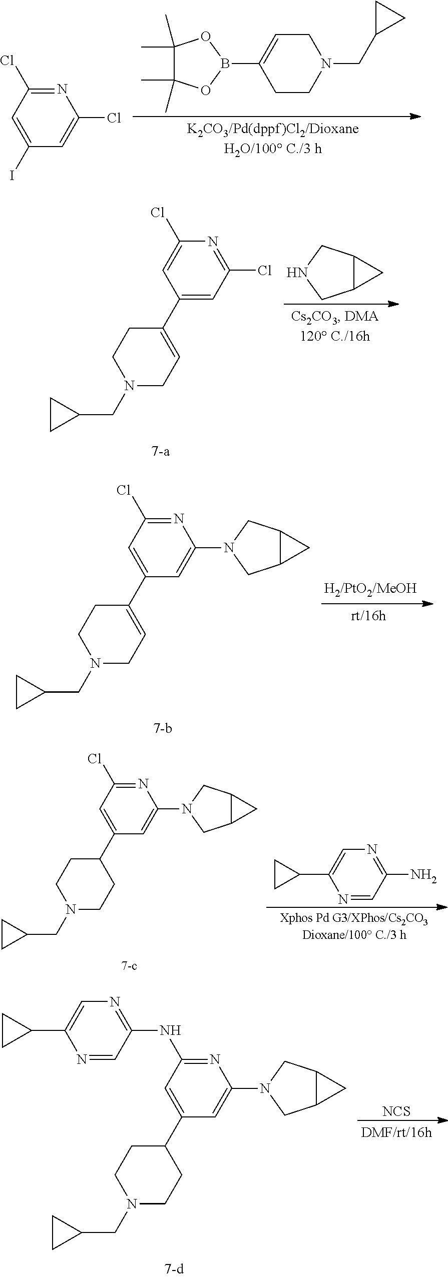

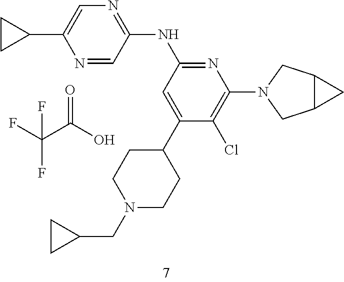

### Step 1: 2′,6′-Dichloro-1-(cyclopropylmethyl)-3,6-dihydro-2H-4,4′-bipyridine

To a stirred solution of 2,6-dichloro-4iodopyridine (2.00 g, 7.30 mmol, 1 equiv) and 1-(cyclopropylmethyl)-4-(4,4,5,5-tetramethyl-1,3,2-dioxaborolan-2-yl)-3,6-dihydro-2H-pyridine (2.31 g, 8.76 mmol, 1.2 equiv) in dioxane (50 mL): H₂O (5 mL) were added K₂CO₃ (2.02 g, 14.6 mmol, 2 equiv) and Pd(dppf)Cl₂CH₂Cl₂ (0.590 g, 0.730 mmol, 0.1 equiv) in portions at room temperature under nitrogen atmosphere. The resulting mixture was stirred for 3 h at 100° C. under nitrogen atmosphere. The reaction mixture was cooled to room temperature and quenched with water (10 mL). The resulting mixture was extracted with EtOAc (3×100 mL). The combined organic layers were washed with brine (2×100 mL) and dried over anhydrous Na₂SO₄. After filtration, the filtrate was concentrated under reduced pressure. The residue was purified by silica gel column chromatography to afford 2′,6′-dichloro-1-(cyclopropylmethyl)-3,6-dihydro-2H-4,4′-bipyridine (1.80 g, 87%) as a brown oil. LC-MS: (ES, m/z): [M+H]=283.

### Step 2: 3-(6-Chloro-1′-(cyclopropylmethyl)-1′,2′,3′,6′-tetrahydro-[4,4′-bipyridin]-2-yl)-3-azabicyclo[3.1.0]hexane

To a stirred solution of 2′,6′-dichloro-1-(cyclopropylmethyl)-3,6-dihydro-2H-4,4′-bipyridine (1.80 g, 6.36 mmol, 1 equiv) and 3-azabicyclo [3.1.0] hexane (0.630 g, 7.63 mmol, 1.2 equiv) in DMA (50 mL) was added Cs₂CO₃ (6.21 g, 19.1 mmol, 3 equiv) in portions at room temperature. The resulting mixture was stirred for 16 h at 120° C. The reaction mixture was cooled to room temperature and quenched with water (20 mL). The resulting mixture was extracted with EtOAc (3×100 mL). The combined organic layers were washed with water (3×100 mL) and dried over anhydrous Na₂SO₄. After filtration, the filtrate was concentrated under reduced pressure. The residue was purified by silica gel column chromatography to afford 3-(6-chloro-1′-(cyclopropylmethyl)-1′,2′,3′,6′-tetrahydro-[4,4′-bipyridin]-2-yl)-3-azabicyclo[3.1.0]hexane (1.00 g, 48%) as a yellow oil. LC-MS: (ES, m/z): [M+H]⁺=330.

### Step 3: 3-(6-Chloro-4-(1-(cyclopropylmethyl)piperidin-4-yl)pyridin-2-yl)-3-azabicyclo[3.1.0]hexane

To a stirred solution of 3-(6-chloro-1′-(cyclopropylmethyl)-1′,2′,3′,6′-tetrahydro-[4,4′-bipyridin]-2-yl)-3-azabicyclo[3.1.0]hexane (700 mg, 2.12 mmol, 1 equiv) in MeOH (20 mL) was added PtO₂ (337 mg, 1.49 mmol, 0.7 equiv) in portions at room temperature under hydrogen atmosphere. The resulting mixture was stirred for 16 h at room temperature under hydrogen atmosphere. The resulting mixture was filtered and the filter cake was washed with MeOH (2×50 mL). The filtrate was concentrated under reduced pressure to afford 3-(6-chloro-4-(1-(cyclopropylmethyl)piperidin-4-yl)pyridin-2-yl)-3-azabicyclo[3.1.0]hexane (300 mg, 43%) as a brown oil, which was used in the next step directly without further purification. LC-MS: (ES, m/z): [M+H]=332.

### Step 4: N-(6-(3-Azabicyclo[3.1.0]hexan-3-yl)-4-(1-(cyclopropylmethyl)piperidin-4-yl)pyridin-2-yl)-5-cyclopropylpyrazin-2-amine

To a stirred solution of 3-(6-chloro-4-(1-(cyclopropylmethyl)piperidin-4-yl)pyridin-2-yl)-3-azabicyclo[3.1.0]hexane (300 mg, 0.904 mmol, 1 equiv) and 5-cyclopropylpyrazin-2-amine (147 mg, 1.09 mmol, 1.2 equiv) in dioxane (20 mL) were added XPhos Pd G3 (76.5 mg, 0.090 mmol, 0.1 equiv), XPhos (86.2 mg, 0.181 mmol, 0.2 equiv), and Cs₂CO₃ (589 mg, 1.81 mmol, 2 equiv) in portions at room temperature under nitrogen atmosphere. The resulting mixture was stirred for 3 h at 100° C. under nitrogen atmosphere. The reaction mixture was cooled to room temperature and quenched with water (20 mL). The resulting mixture was extracted with EtOAc (3×50 mL). The combined organic layers were washed with water (2×50 mL) and dried over anhydrous Na₂SO₄. After filtration, the filtrate was concentrated under reduced pressure. The residue was purified by silica gel column chromatography to afford N-(6-(3-azabicyclo[3.1.0]hexan-3-yl)-4-(1-(cyclopropylmethyl)piperidin-4-yl)pyridin-2-yl)-5-cyclopropylpyrazin-2-amine (180 mg, 46%) as a yellow oil. LC-MS (ES, m/z): [M+H]⁺=431.

### Step 5: N-(6-(3-Azabicyclo[3.1.0]hexan-3-yl)-5-chloro-4-(1-(cyclopropylmethyl)piperidin-4-yl)pyridin-2-yl)-5-cyclopropylpyrazin-2-amine TFA salt (Compound 7)

To a stirred solution of N-(6-(3-azabicyclo[3.1.0]hexan-3-yl)-4-(1-(cyclopropylmethyl)piperidin-4-yl)pyridin-2-yl)-5-cyclopropylpyrazin-2-amine (100 mg, 0.232 mmol, 1 equiv) in DMF (5 mL) was added NCS (31 mg, 0.232 mmol, 1 equiv) in portions at room temperature. The resulting mixture was stirred for 16 h at room temperature. The reaction was quenched by the addition of water (1 mL) at room temperature. The resulting mixture was extracted with EtOAc (2×10 mL). The combined organic layers were washed with water (1×10 mL) and dried over anhydrous Na₂SO₄. After filtration, the filtrate was concentrated under reduced pressure. The crude product (50 mg) was purified by Prep-HPLC with the following conditions (Column: XBridge Prep Phenyl OBD Column 19*250 mm, 5 m; Mobile Phase A: Water (0.1% FA), Mobile Phase B: MEOH; Flow rate: 60 mL/min; Gradient: 2% B to 22% B in 10 min; Wave Length: 254 nm/220 nm) to afford N-(6-(3-azabicyclo[3.1.0]hexan-3-yl)-5-chloro-4-(1-(cyclopropylmethyl)piperidin-4-yl)pyridin-2-yl)-5-cyclopropylpyrazin-2-amine TFA salt (Compound 7) (10.4 mg, 7%) as a yellow solid. LC-MS: (ES, m/z): [M+H]=465. ¹H-NMR (400 MHz, CD₃OD): δ 8.78 (s, 1H), 8.18 (s, 1H), 6.75 (s, 1H), 4.17 (d, J=9.6 Hz, 2H), 3.81 (d, J=12.4 Hz, 2H), 3.67 (d, J=10.0 Hz, 2H), 3.46-3.36 (m, 1H), 3.18 (t, J=12.0 Hz, 2H), 3.08 (d, J=7.2 Hz, 2H), 2.23-1.94 (m, 5H), 1.70 (t, J=4.0 Hz, 2H), 1.22-1.11 (m, 1H), 1.06-0.93 (m, 4H), 0.83-0.67 (m, 3H), 0.52-0.37 (m, 3H). ¹⁹F-NMR (400 MHz, DMSO-d₆): δ −74.53.

### Example 8: 2-(3-Azabicyclo[3.1.0]hexan-3-yl)-4-(1-(cyclopropylmethyl)piperidin-4-yl)-6-((5-cyclopropylpyrazin-2-yl)amino)nicotinonitrile formic acid salt (Compound 8)

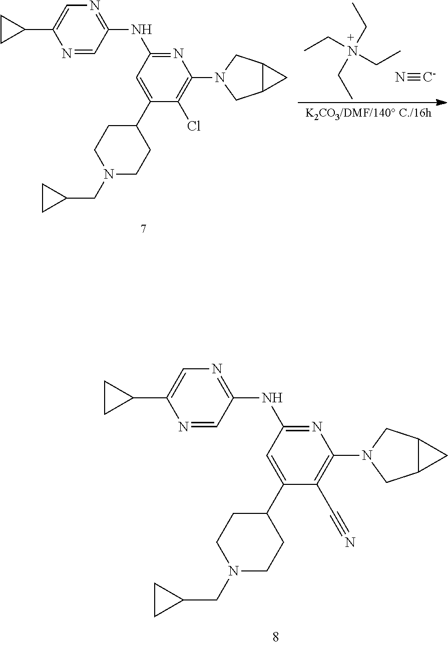

To a stirred solution of N-(6-(3-azabicyclo[3.1.0]hexan-3-yl)-5-chloro-4-(1-(cyclopropylmethyl)piperidin-4-yl)pyridin-2-yl)-5-cyclopropylpyrazin-2-amine (Compound 7) (100 mg, 0.215 mmol, 1 equiv) and cyanide tetraethylammonium (101 mg, 0.645 mmol, 3 equiv) in DMF (5 mL) was added K₂CO₃ (89.2 mg, 0.645 mmol, 3 equiv) in portions at room temperature. The resulting mixture was stirred for 16 h at 140° C. The reaction mixture was cooled to room temperature and quenched with water (5 mL). The resulting mixture was extracted with EtOAc (2×20 mL). The combined organic layers were washed with brine (2×20 mL) and dried over anhydrous Na₂SO₄. After filtration, the filtrate was concentrated under reduced pressure. The crude product (40 mg) was purified by Prep-HPLC with the following conditions (Column: Sunfire prep C18 column, 30*150 mm, 5 m; Mobile Phase A: Water (0.1% FA), Mobile Phase B: MeOH—HPLC; Flow rate: 60 mL/min; Gradient: 32% B to 50% B in 10 min; Wave Length: 254 nm/220 nm) to afford 2-(3-azabicyclo[3.1.0]hexan-3-yl)-4-(1-(cyclopropylmethyl)piperidin-4-yl)-6-((5-cyclopropylpyrazin-2-yl)amino)nicotinonitrile formic acid salt (Compound 8) (2 mg, 2%) as a white solid. LC-MS: (ES, m/z): [M+H]=456. ¹H-NMR (400 MHz, DMSO-d6): δ 9.87 (s, 1H), 9.08 (s, 1H), 8.27 (s, 2H), 8.19 (s, 1H), 6.50 (s, 1H), 3.96 (d, J=12.0 Hz, 2H), 3.62 (d, J=10.4 Hz, 2H), 3.01 (d, J=11.2 Hz, 2H), 2.55 (t, J=12.4 Hz, 1H), 2.13 (d, J=6.4 Hz, 2H), 2.07-1.99 (m, 1H), 1.92 (t, J=11.2 Hz, 2H), 1.73-1.59 (m, 4H), 1.55-1.41 (m, 2H), 0.90-0.60 (m, 6H), 0.38 (d, J=7.2 Hz, 2H), 0.08 (d, J=3.2 Hz, 1H), 0.01 (d, J=4.8 Hz, 2H).

### Example 9: N-(6-(3-Azabicyclo[3.1.0]hexan-3-yl)-5-chloro-4-(1-(cyclopropylmethyl)piperidin-4-yl)pyridin-2-yl)-5-cyclopropylpyrazin-2-amine TFA salt (Compound 9)

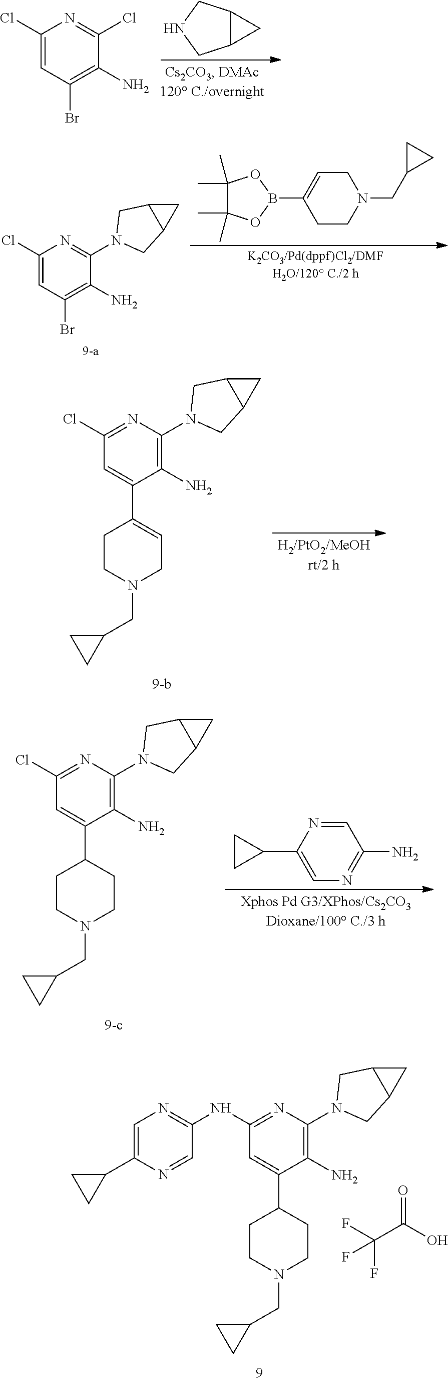

### Step 1: 2-(3-Azabicyclo[3.1.0]hexan-3-yl)-4-bromo-6-chloropyridin-3-amine

A mixture of 4-bromo-2,6-dichloropyridin-3-amine (4 g, 16.536 mmol), 3-azabicyclo[3.1.0]hexane (1.37 g, 16.536 mmol) and Cs₂CO₃ (16.16 g, 49.608 mmol) in N,N-dimethylacetamide (20 mL) was stirred overnight at 120° C. under nitrogen atmosphere. The reaction was cooled to room temperature and quenched with sat. NH₄Cl (aq.). The resulting mixture was extracted with EtOAc (3×200 mL). The combined organic layers were washed with brine (2×400 mL) and dried over anhydrous Na₂SO₄. After filtration, the filtrate was concentrated under reduced pressure. The residue was purified by silica gel column chromatography to afford 2 -(3-azabicyclo[3.1.0]hexan-3-yl)-4-bromo-6-chloropyridin-3-amine (1.1 g, 23%) as a purple oil. LC-MS: (ES, m/z): [M+H]=288.

### Step 2: 2-(3-Azabicyclo[3.1.0]hexan-3-yl)-6-chloro-1′-(cyclopropylmethyl)-1′,2′,3′,6′-tetrahydro-[4,4′-bipyridin]-3-amine

A mixture of 2-(3-azabicyclo[3.1.0]hexan-3-yl)-4-bromo-6-chloropyridin-3-amine (0.3 g, 1.040 mmol), 1-(cyclopropylmethyl)-4-(4,4,5,5-tetramethyl-1,3,2-dioxaborolan-2-yl)-3,6-dihydro-2H-pyridine (0.33 g, 1.248 mmol), K₂CO₃ (0.43 g, 3.120 mmol) and Pd(dppf)Cl₂ (0.08 g, 0.104 mmol) in DMF (6 mL) and H₂O (0.6 mL) was stirred for 2 h at 120° C. under nitrogen atmosphere. The reaction mixture was cooled to room temperature and quenched with water (100 mL). The resulting mixture was extracted with EtOAc (3×100 mL). The combined organic layers were washed with water (3×100 mL) and dried over anhydrous Na₂SO₄. After filtration, the filtrate was concentrated under reduced pressure. The residue was purified by silica gel column chromatography to afford 2-(3-azabicyclo[3.1.0]hexan-3-yl)-6-chloro-1′-(cyclopropylmethyl)-1′,2′,3′,6′-tetrahydro-[4,4′-bipyridin]-3-amine (0.25 g, 70%) as a brown oil. LC-MS: (ES, m/z): [M+H]⁺=345.

### Step 3: 2-(3-Azabicyclo[3.1.0]hexan-3-yl)-6-chloro-4-(1-(cyclopropylmethyl)piperidin-4-yl)pyridin-3-amine

To a stirred solution of 2-(3-azabicyclo[3.1.0]hexan-3-yl)-6-chloro-1′-(cyclopropylmethyl)-1′,2′,3′,6′-tetrahydro-[4,4′-bipyridin]-3-amine (250 mg, 0.725 mmol) in MeOH (20 mL) was added PtO₂ (115.22 mg, 0.507 mmol) in portions at room temperature. The resulting mixture was stirred for 2 h at room temperature under hydrogen atmosphere. The resulting mixture was filtered and the filter cake was washed with MeOH (2×50 mL). The filtrate was concentrated under reduced pressure to afford 2-(3-azabicyclo[3.1.0]hexan-3-yl)-6-chloro-4-(1-(cyclopropylmethyl)piperidin-4-yl)pyridin-3-amine (150 mg, 60%) as a red solid. LC-MS: (ES, m/z): [M+H]⁺=347.

### Step 4: 6-(3-azabicyclo[3.1.0]hexan-3-yl)-4-(1-(cyclopropylmethyl)piperidin-4-yl)-N2-(5-cyclopropylpyrazin-2-yl)pyridine-2,5-diamine 2,2,2-trifluoroacetate

To a stirred solution of 2-(3-azabicyclo[3.1.0]hexan-3-yl)-6-chloro-4-(1-(cyclopropylmethyl)piperidin-4-yl)pyridin-3-amine (150 mg, 0.432 mmol) in dioxane (20 mL) were added Cs₂CO₃ (704 mg, 2.160 mmol), 5-cyclopropylpyrazin-2-amine (59 mg, 0.432 mmol), Xphos Pd G3 (37 mg, 0.043 mmol) and XPhos (31 mg, 0.065 mmol) in portions at room temperature. The resulting mixture was stirred for 3 h at 100° C. under nitrogen atmosphere. The resulting mixture was concentrated under reduced pressure. The residue was purified by silica gel column chromatography and then purified by Prep-HPLC with the following conditions (Column: Sunfire prep C18 column, 30*150 mm, 5 m; Mobile Phase A: Water (0.05% TFA), Mobile Phase B: ACN; Flow rate: 60 mL/min; Gradient: isocratic 5% B to 25% B in 10 min; Wave Length: 254 nm/220 nm) to afford 6-(3-azabicyclo[3.1.0]hexan-3-yl)-4-(1-(cyclopropylmethyl)piperidin-4-yl)-N2-(5-cyclopropylpyrazin-2-yl)pyridine-2,5-diamine 2,2,2-trifluoroacetate (Compound 9) (19 mg, 8%) as a light brown solid. LC-MS (ES, m/z): [M+H]⁺=446. ¹H-NMR (400 MHz, DMSO-d₆): δ 9.81-9.23 (m, 2H), 8.98-8.72 (m, 1H), 8.11-8.01 (m, 1H), 6.78-6.52 (m, 1H), 3.69-3.45 (m, 4H), 3.34 (d, J=9.6 Hz, 1H), 3.24 (d, J=9.6 Hz, 1H), 2.97-2.85 (m, 4H), 2.05-1.80 (m, 3H), 1.79-1.62 (m, 3H), 1.55-1.45 (m, 2H), 1.06-0.90 (m, 1H), 0.85-0.72 (m, 4H), 0.61-0.48 (m, 3H), 0.48-0.39 (m, 1H), 0.33-0.20 (m, 2H). ¹⁹F-NMR (376 MHz, DMSO-d₆): δ −74.52.

### Example 10: 5-Cyclopropyl-N-(5-fluoro-6-morpholino-4-(piperazin-1-yl)pyridin-2-yl)pyrazin-2-amine (Compound 10)

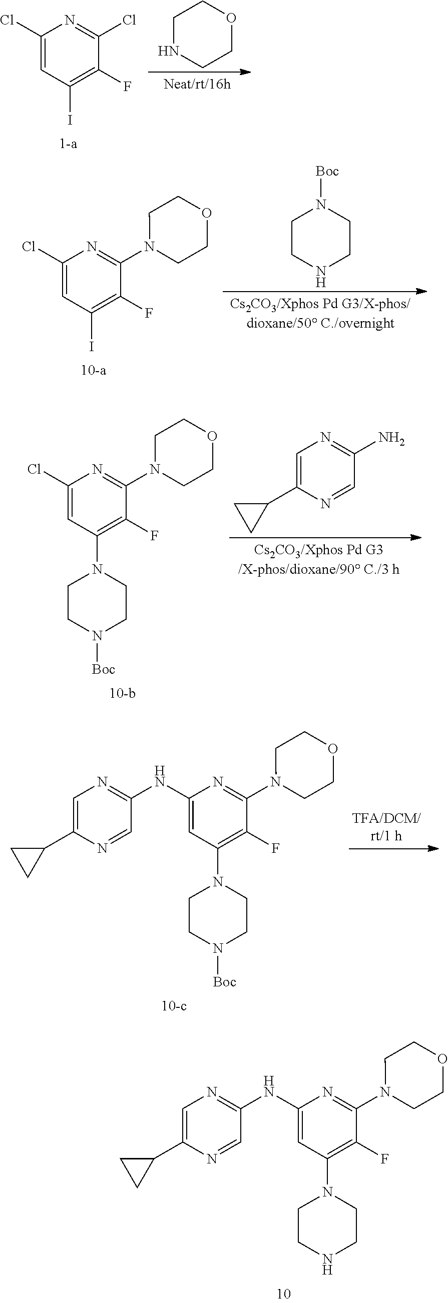

Compound 1-a was synthesized as described in Example 1.

### Step 1: 4-(6-Chloro-3-fluoro-4iodopyridin-2-yl)morpholine

A solution of 2,6-dichloro-3-fluoro-4iodopyridine (5 g, 17.131 mmol, 1 equiv) in morpholine (8 mL) was stirred for 16 h at room temperature under nitrogen atmosphere. The reaction was quenched by the addition of water (50 mL). The resulting mixture was extracted with EtOAc (3×100 mL). The combined organic layers were washed with water (2×40 mL) and dried over anhydrous Na₂SO₄. After filtration, the filtrate was concentrated under reduced pressure. The residue was purified by silica gel column chromatography to afford 4-(6-chloro-3-fluoro-4-iodopyridin-2-yl)morpholine (5 g, 85%) as a white solid. LC-MS: (ES, m/z): [M+H]⁺=343.

### Step 2: tert-Butyl 4-(6-chloro-3-fluoro-2-morpholinopyridin-4-yl)piperazine-1-carboxylate

A mixture of 4-(6-chloro-3-fluoro-4iodopyridin-2-yl)morpholine (2 g, 5.839 mmol, 1 equiv), tert-butyl piperazine-1-carboxylate (1.09 g, 5.839 mmol, 1 equiv), Cs₂CO₃ (1.90 g, 5.839 mmol, 1 equiv), Xphos Pd G3 (0.49 g, 0.584 mmol, 0.1 equiv) and X-phos (0.56 g, 1.168 mmol, 0.2 equiv) in dioxane (20 mL) was stirred overnight at 50° C. under nitrogen atmosphere. The reaction was quenched by the addition of ice water (50 mL) at room temperature. The resulting mixture was extracted with EtOAc (3×100 mL). The combined organic layers were washed with water (2×50 mL) and dried over anhydrous Na₂SO₄. After filtration, the filtrate was concentrated under reduced pressure. The residue was purified by silica gel column chromatography to afford tert-butyl 4-(6-chloro-3-fluoro-2-morpholinopyridin-4-yl)piperazine-1-carboxylate (1.2 g, 51%) as a brown oil. LC-MS: (ES, m/z): [M+H]⁺=401.

### Step 3: tert-Butyl 4-(6-((5-cyclopropylpyrazin-2-yl)amino)-3-fluoro-2-morpholinopyridin-4-yl)piperazine-1-carboxylate

To a stirred solution of tert-butyl 4-(6-chloro-3-fluoro-2-morpholinopyridin-4-yl)piperazine-1-carboxylate (120 mg, 0.299 mmol, 1 equiv) and 5-cyclopropylpyrazin-2-amine (40 mg, 0.299 mmol, 1 equiv) in dioxane (5 mL) were added XPhos Pd G3 (25 mg, 0.030 mmol, 0.1 equiv), XPhos (14 mg, 0.030 mmol, 0.1 equiv) and Cs₂CO₃ (195 mg, 0.598 mmol, 2 equiv) in portions at room temperature under nitrogen atmosphere. The resulting mixture was stirred for 3 h at 90° C. under nitrogen atmosphere. The reaction mixture was cooled to room temperature and quenched with water (5 mL). The resulting mixture was extracted with EtOAc (2×20 mL). The combined organic layers were washed with brine (1×20 mL) and dried over anhydrous Na₂SO₄. After filtration, the filtrate was concentrated under reduced pressure. The residue was purified by silica gel column chromatography to afford tert-butyl 4-(6-((5-cyclopropylpyrazin-2-yl)amino)-3-fluoro-2-morpholinopyridin-4-yl)piperazine-1-carboxylate (100 mg, 67%) as a yellow oil. LC-MS: (ES, m/z): [M+H]⁺=500.

### Step 4: 5-Cyclopropyl-N-(5-fluoro-6-morpholino-4-(piperazin-1-yl)pyridin-2-yl)pyrazin-2-amine (Compound 10)

A solution of tert-butyl 4-(6-((5-cyclopropylpyrazin-2-yl)amino)-3-fluoro-2-morpholinopyridin-4-yl)piperazine-1-carboxylate (100 mg, 0.200 mmol, 1 equiv) in TFA (1 mL):DCM (5 mL) was stirred for 1 h at room temperature. The resulting mixture was concentrated under reduced pressure. The mixture was neutralized to pH 7 with NH₃·H₂O. The crude product (80 mg) was purified by Prep-HPLC with the following conditions (Column: YMC-Actus Triart C18 ExRS 30*150 mm, 5 m; Mobile Phase A: Water(10 mmol/L NH₄HCO₃+0.05% NH₃H₂O), Mobile Phase B: ACN; Flow rate: 60 mL/min; Gradient: 29% B to 46% B in 10 min; Wave Length: 254 nm/220 nm) to afford 5-cyclopropyl-N-(5-fluoro-6-morpholino-4-(piperazin-1-yl)pyridin-2-yl)pyrazin-2-amine (Compound 10) (48 mg, 60%) as a white solid. LC-MS: (ES, m/z): [M+H]+=400. ¹H-NMR (400 MHz, DMSO-d6) δ 9.36 (s, 1H), 8.97 (d, J=1.6 Hz, 1H), 8.15 (d, J=1.4 Hz, 1H), 6.67 (d, J=4.8 Hz, 1H), 3.78-3.67 (m, 4H), 3.26 (d, J=9.4 Hz, 4H), 3.02 (dd, J=6.4, 3.4 Hz, 4H), 2.88-2.76 (m, 4H), 2.11-2.01 (m, 1H), 0.95-0.87 (m, 2H), 0.83 (m, 2H).

### Example 11: LZK and DLK Inhibition Assay

The kinases were produced in HEK-293 cells and subsequently tagged with DNA for qPCR detection. Streptavidin-coated magnetic beads were treated with biotinylated small molecule ligands for 30 minutes at room temperature to generate affinity resins for the kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining LZK and DLK kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (lx PBS, 0.05% Tween 20). The beads were then re-suspended in elution buffer (1×PBS, 0.05% Tween 20, 0.5 μM nonbiotinylated affinity ligand) and incubated at room temperature with shaking for 30 minutes. The kinase concentration in the eluates was measured by qPCR.

Representative data for exemplary compounds disclosed herein is presented in Table 1.

TABLE 1

| Compound | LZK IC₅₀ | DLK IC₅₀ |
|---|---|---|
| 1 | +++ | +++ |
| 2 | ++ | +++ |
| 3 | ++ | ++ |
| 4 | + | + |
| 5 | ++ | + |
| 6 | + | + |
| 7 | + | + |
| 8 | +++ | +++ |
| 9 | +++ | +++ |
| 10 | +++ | NT |

where ‘+++’ means IC₅₀ ≤ 0.1 μM; where ‘++’ means 0.1 μM < IC₅₀ ≤ 1 μM; where ‘+’ means 1 μM < IC₅₀ ≤ 10 μM; NT means not tested.

What is claimed is:

1. A compound of Formula (I), or a pharmaceutically acceptable salt or solvate thereof: 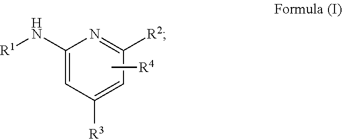
wherein:
R¹ is a 6-membered heteroaryl ring optionally substituted with one, two, or three R⁵;
R² is —N(R¹⁰)(R¹¹), C₂₋₉heterocycloalkyl, —CH₂—C₂₋₉heterocycloalkyl, C₃₋₆cycloalkyl, or —CH₂—C₃₋₆cycloalkyl, wherein C₂₋₉heterocycloalkyl, —CH₂—C₂₋₉heterocycloalkyl, C₃₋₆cycloalkyl, and —CH₂—C₃₋₆cycloalkyl are optionally substituted with one, two, or three R⁶;
R³ is C₃₋₆cycloalkyl, C₂₋₉heterocycloalkyl, C₆₋₁₀aryl, or C₁₋₉heteroaryl, wherein each C₃₋₆cycloalkyl, C₂₋₉heterocycloalkyl, C₆₋₁₀aryl, and C₁₋₉heteroaryl are optionally substituted with one, two, or three R⁷;
R⁴ is selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀ aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R⁵ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R⁶ is independently selected from halogen, oxo, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R⁷ is independently selected from halogen, —CN, C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —N(R¹²)C(O)R¹³, —S(O)₂R¹³, —S(O)₂N(R¹⁰)(R¹¹)—, —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₁₋₆haloalkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

2. The compound of claim 1, or a pharmaceutically acceptable salt or solvate thereof, having the structure of Formula (Ia):

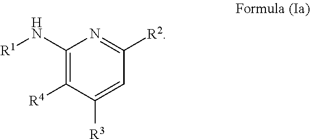

3. The compound of claim 1, or a pharmaceutically acceptable salt or solvate thereof, having the structure of Formula (Ib):

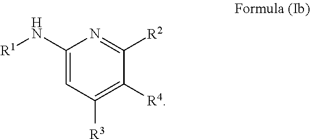

4. The compound of any one of claims 1-3, or a pharmaceutically acceptable salt or solvate thereof, wherein R² is C₂₋₉heterocycloalkyl optionally substituted with one, two, or three R⁶.

5. The compound of any one of claims 1-4, or a pharmaceutically acceptable salt or solvate thereof, wherein R² is selected from 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl, wherein 3-azabicyclo[3.1.0]hexanyl, 3-azabicyclo[3.2.0]heptanyl, 3-azabicyclo[3.1.1]heptanyl, 2-oxa-5-azabicyclo[2.2.1]heptanyl, morpholinyl, piperidinyl, pyrrolidinyl, azetidinyl, and piperazinyl are optionally substituted with one, two, or three R⁶.

6. The compound of any one of claims 1-5, or a pharmaceutically acceptable salt or solvate thereof, wherein each R⁶ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆cycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

7. The compound of any one of claims 1-6, or a pharmaceutically acceptable salt or solvate thereof, wherein each R⁶ is independently selected from halogen and C₁₋₆haloalkyl.

8. The compound of any one of claims 1-7, or a pharmaceutically acceptable salt or solvate thereof, wherein R² is selected from

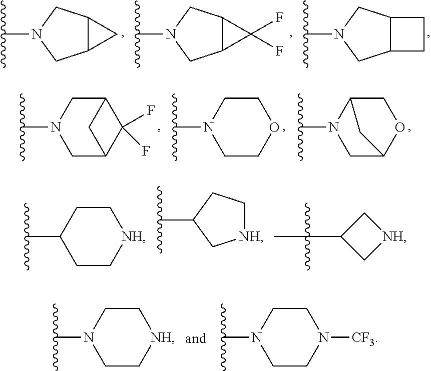

9. The compound of any one of claims 1-8, or a pharmaceutically acceptable salt or solvate thereof, wherein R² is selected from

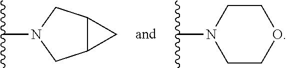 [SMILES: *N1CC2CC2C1 ; *N1CCOCC1]

10. The compound of any one of claims 1-9, or a pharmaceutically acceptable salt or solvate thereof, wherein R³ is C₂₋₉heterocycloalkyl optionally substituted with one, two, or three R⁷.

11. The compound of any one of claims 1-10, or a pharmaceutically acceptable salt or solvate thereof, wherein R³ is selected from 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl, wherein 2,5-diazabicyclo[2.2.1]heptane, 2-oxa-5-azabicyclo[2.2.1]heptane, 2-oxa-7-azaspiro[3.5]nonanyl, morpholinyl, piperidinyl, 1,2,3,6-tetrahydropyridinyl, pyrrolidinyl, azetidinyl, piperazinyl, and tetrahydropyranyl are optionally substituted with one, two, or three R⁷.

12. The compound of any one of claims 1-11, or a pharmaceutically acceptable salt or solvate thereof, wherein each R⁷ is independently selected from halogen, C₁₋₆alkyl, C₁₋₆haloalkyl, C₃₋₆cycloalkyl, and —CH₂—C₃₋₆cycloalkyl, wherein C₁₋₆alkyl, C₁₋₆haloalkyl, C₃₋₆cycloalkyl, and —CH₂—C₃₋₆cycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

13. The compound of any one of claims 1-12, or a pharmaceutically acceptable salt or solvate thereof, wherein each R⁷ is independently selected from halogen, unsubstituted C₁₋₆alkyl, and unsubstituted —CH₂—C₃₋₆cycloalkyl.

14. The compound of any one of claims 1-13, or a pharmaceutically acceptable salt or solvate thereof, wherein R³ is selected from

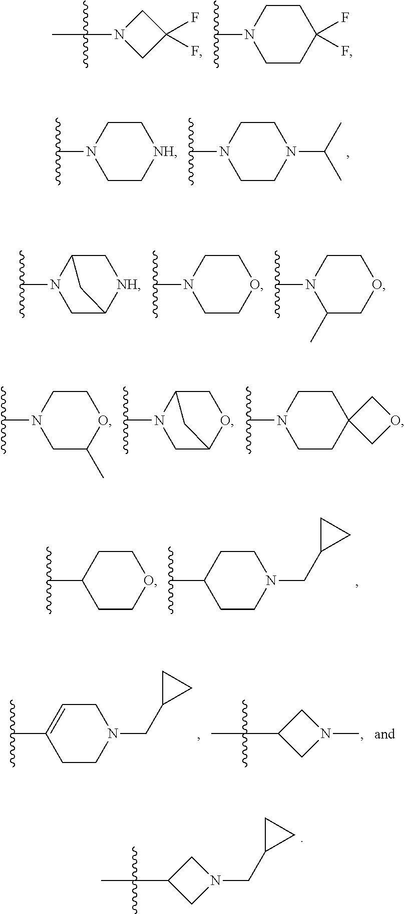

15. The compound of any one of claims 1-14, or a pharmaceutically acceptable salt or solvate thereof, wherein R³ is selected

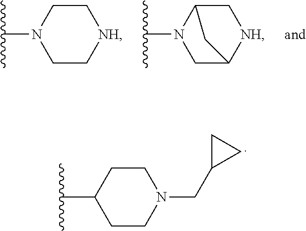

16. The compound of any one of claims 1-15, or a pharmaceutically acceptable salt or solvate thereof, wherein R⁴ is selected from halogen, —CN, C₁₋₆alkyl, and C₁₋₆haloalkyl, wherein C₁₋₆alkyl and C₁₋₆haloalkyl are optionally substituted with one, two, or three groups selected from halogen, C₁₋₆alkyl, C₁₋₆haloalkyl, —OR¹⁰, and —N(R¹⁰)(R¹¹).

17. The compound of any one of claims 1-16, or a pharmaceutically acceptable salt or solvate thereof, wherein R⁴ is halogen.

18. The compound of any one of claims 1-16, or a pharmaceutically acceptable salt or solvate thereof, wherein R⁴ is —CN.

19. The compound of any one of claims 1-18, or a pharmaceutically acceptable salt or solvate thereof, wherein R¹ is pyrazinyl or pyridinyl, wherein the pyrazinyl or pyridinyl are optionally substituted with one, two, or three R⁵.

20. The compound of any one of claims 1-19, or a pharmaceutically acceptable salt or solvate thereof, wherein R¹ is pyrazinyl or pyridinyl, wherein the pyrazinyl are pyridinyl are optionally substituted with one R⁵.

21. The compound of any one of claims 1-20, or a pharmaceutically acceptable salt or solvate thereof, wherein each R⁵ is independently selected from halogen, C₁₋₆alkyl, C₁₋₆haloalkyl, C₃₋₆cycloalkyl, and C₂₋₉heterocycloalkyl, wherein C₁₋₆alkyl, C₁₋₆haloalkyl, C₃₋₆cycloalkyl, and C₂₋₉heterocycloalkyl are optionally substituted with one, two, or three groups selected from halogen, C₁₋₆alkyl, C₁₋₆haloalkyl, —OR¹⁰, —N(R¹⁰)(R¹¹), and —C(O)OR¹⁰.

22. The compound of any one of claims 1-21, or a pharmaceutically acceptable salt or solvate thereof, wherein R¹ is pyrazinyl or pyridinyl, wherein the pyrazinyl or pyridinyl are substituted with one R⁵ and R⁵ is cyclopropyl.

23. The compound of any one of claims 1-22, or a pharmaceutically acceptable salt or solvate thereof, wherein R¹ is

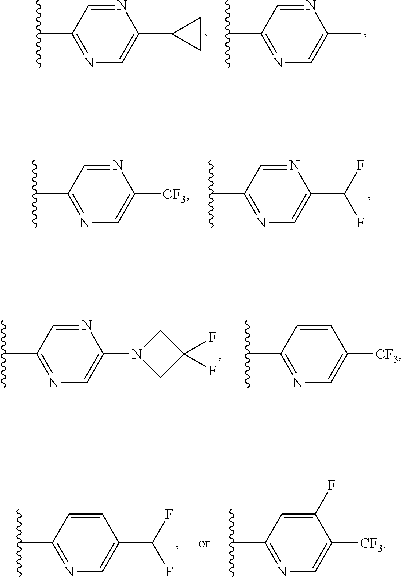

24. The compound of any one of claims 1-23, or a pharmaceutically acceptable salt or solvate thereof, wherein R¹ is

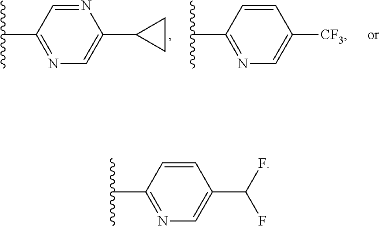

25. A compound selected from: 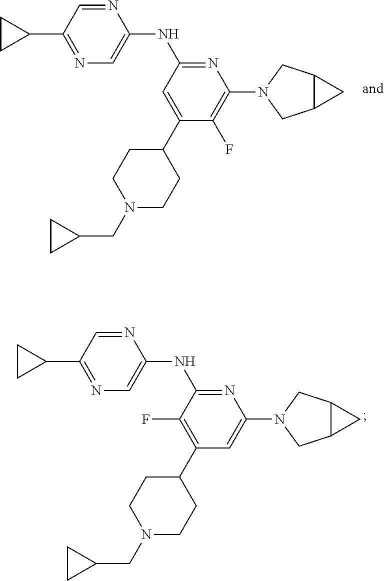 [SMILES: Fc1c(C2CCN(CC3CC3)CC2)cc(Nc2cnc(C3CC3)cn2)nc1N1CC2CC2C1 ; Fc1c(C2CCN(CC3CC3)CC2)cc(N2CC3CC3C2)nc1Nc1cnc(C2CC2)cn1]
or a pharmaceutically acceptable salt or solvate thereof.

26. A compound selected from: 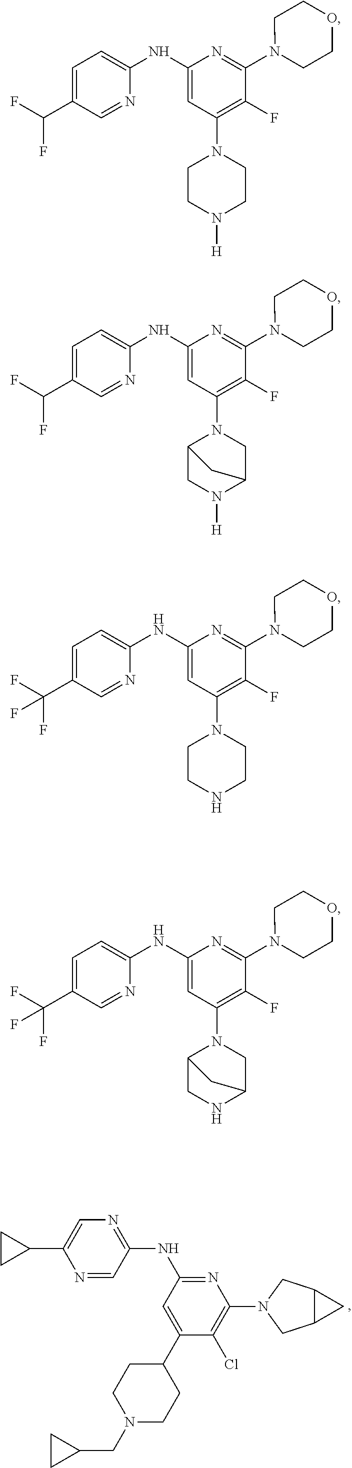 [SMILES: Fc1c(N2CCNCC2)cc(Nc2ccc(C(F)F)cn2)nc1N1CCOCC1 ; Fc1c(N2CC3CC2CN3)cc(Nc2ccc(C(F)F)cn2)nc1N1CCOCC1 ; Fc1c(N2CCNCC2)cc(Nc2ccc(C(F)(F)F)cn2)nc1N1CCOCC1 ; Fc1c(N2CC3CC2CN3)cc(Nc2ccc(C(F)(F)F)cn2)nc1N1CCOCC1 ; Clc1c(C2CCN(CC3CC3)CC2)cc(Nc2cnc(C3CC3)cn2)nc1N1CC2CC2C1] 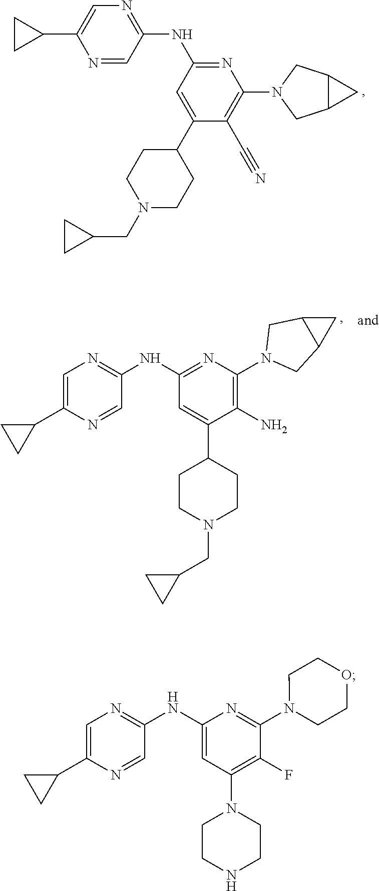 [SMILES: N#Cc1c(C2CCN(CC3CC3)CC2)cc(Nc2cnc(C3CC3)cn2)nc1N1CC2CC2C1 ; Nc1c(C2CCN(CC3CC3)CC2)cc(Nc2cnc(C3CC3)cn2)nc1N1CC2CC2C1 ; Fc1c(N2CCNCC2)cc(Nc2cnc(C3CC3)cn2)nc1N1CCOCC1]
or a pharmaceutically acceptable salt or solvate thereof.

27. A pharmaceutical composition comprising a compound of any one of claims 1-26, or a pharmaceutically acceptable salt or solvate thereof, and at least one pharmaceutically acceptable excipient.

28. A method of treating cancer in a mammal, comprising administering to the mammal a compound of any one of claims 1-26, or a pharmaceutically acceptable salt or solvate thereof.

29. The method of claim 28, wherein the cancer is a cancer with LZK alterations.

30. The method of claim 28 or claim 29, wherein the cancer is selected from head and neck squamous cell carcinoma and esophageal squamous cell carcinoma.
